(19) United States
(12) Patent Application Publication
(10) Pub. No.: US 2020/0274077 A1
(43) Pub. Date: Aug. 27, 2020

(54) PHOTOELECTRIC CONVERSION ELEMENT AND IMAGING APPARATUS

(71) Applicants: SONY CORPORATION, Tokyo (JP); SONY SEMICONDUCTOR SOLUTIONS CORPORATION, Kanagawa (JP)

(72) Inventors: Yasuharu UJIIE, Kanagawa (JP); Yosuke SAITO, Tokyo (JP); Yuta HASEGAWA, Kanagawa (JP); Hideaki MOGI, Kanagawa (JP); Osamu ENOKI, Kanagawa (JP); Yuki NEGISHI, Kanagawa (JP)

(73) Assignees: SONY CORPORATION, Tokyo (JP); SONY SEMICONDUCTOR SOLUTIONS CORPORATION, Kanagawa (JP)

(21) Appl. No.: 16/761,578

(22) PCT Filed: Oct. 30, 2018
(86) PCT No.: PCT/JP2018/040216

§ 371 Date: May 5, 2020

(30) Foreign Application Priority Data
Nov. 8, 2017 (JP) 2017-215824

(51) Int. Cl. H01L 51/00; H01L 27/30; H01L 27/28; H01L 51/42; H01L 51/44; C07D 495/04

(52) U.S. Cl. CPC H01L 51/0074; H01L 27/307; H01L 27/286; H01L 27/14647; H01L 51/442; C07D 495/04; H01L 51/4253

(57) ABSTRACT

A photoelectric conversion element of the present disclosure includes: a first electrode: a second electrode opposed to the first electrode; and an organic layer provided between the first electrode and the second electrode, and including an organic photoelectric conversion layer, and at least one layer included in the organic layer is formed including at least one kind of organic semiconductor material represented by a general expression (1).

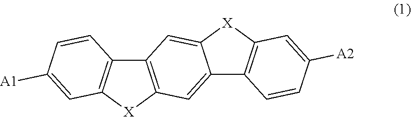

### TECHNICAL FIELD

The present disclosure relates to a photoelectric conversion element using an organic semiconductor material and an imaging apparatus including the same.

### BACKGROUND ART

In recent years, devices using organic thin films have been developed. An organic photoelectric conversion element is one of the devices, and an organic thin-film solar cell and an image sensor (imaging element) each using the organic photoelectric conversion element have been proposed. In addition, providing infrared absorptance characteristics to the organic. photoelectric conversion element makes it possible to achieve high-functionality of a human-detecting sensor, an in-vehicle collision avoidance sensor, and the like.

In the organic photoelectric conversion element, high photoelectric conversion efficiency is desired for any usage. In particular, in an imaging element, in addition to photoelectric conversion efficiency, superior dark current characteristics and superior afterimage characteristics are desired. For this purpose, for example, PTL 1 discloses an organic photoelectric conversion element that includes an organic photoelectric conversion layer, and a hole blocking layer and an electron blocking layer that are disposed between a pair of electrodes with the organic photoelectric conversion layer interposed therebetween and have an adjusted ionization potential.

In addition, PTL 2 discloses a photoelectric conversion element in which a charge blocking layer using a material having high electron mobility is provided between a pair of electrodes and a photoelectric conversion layer disposed between the pair of electrodes.

### CITATION LIST

### Patent Literature

PTL 1: Japanese Unexamined Patent Application Publication No. 2007-88033

PTL 2: Japanese Unexamined Patent Application Publication No. 2009-182096

### SUMMARY OF THE INVENTION

As described above, in a photoelectric conversion element included in an imaging apparatus, in addition to high photoelectric conversion efficiency, superior dark current characteristics and superior afterimage characteristics are desired.

It is therefore desirable to provide a photoelectric conversion element and an imaging apparatus that makes it possible to achieve favorable photoelectric conversion efficiency, superior dark current characteristics, and superior afterimage characteristics.

A photoelectric conversion element according to an embodiment of the present disclosure includes: a first electrode; a second electrode opposed to the first electrode; and an organic layer provided between the first electrode and the second electrode, and including an organic photoelectric conversion layer, and at least one layer included in the organic layer is formed including at least one kind of organic semiconductor material represented by the following general expression (1).

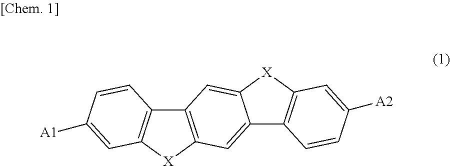

(X is one of an oxygen atom (O), a sulfur atom (S), and a selenium atom (Se), and A1 and A2 are each independently an aryl group, a heteroaryl group, an aryl amino group, a heteroaryl amino group, an aryl group having an aryl amino group as a substituent group, an aryl group having a heteroaryl amino group as a substituent group, a heteroaryl group having an aryl amino group as a substituent group, a heteroaryl group having, a heteroaryl amino group as a substituent group, or a derivative thereof.)

An imaging apparatus according to an embodiment of the present disclosure includes one or a plurality of organic photoelectric converters in each of pixels, and includes the photoelectric conversion element according to the above-described embodiment of the present disclosure as the organic photoelectric converters.

In the photoelectric conversion element according to the embodiment of the present disclosure and the imaging apparatus according to the embodiment of the present disclosure, at least one layer included in the organic layer that is provided between the first electrode and the second electrode and includes the organic photoelectric conversion layer is formed using at least one kind of organic semiconductor material represented by the above-described general expression (1). In the organic semiconductor material represented by the above-described general expression (1), interference with intermolecular interaction in the organic layer is less likely to occur, and a superior orientation property is exhibited in the organic layer. In addition, the organic semiconductor material represented by the general expression (1) forms grains haying a moderate size in the organic layer. This Makes it possible to form an organic layer haying favorable film quality and high carrier transportability.

According to the photoelectric conversion element according to the embodiment of the present disclosure and the imaging apparatus according to the embodiment of the present disclosure, at least one layer included in the organic layer that includes the organic photoelectric conversion layer is formed using at least one kind of organic semiconductor material represented by the above-described general expression (1); therefore, an organic layer having favorable film quality and high carrier transportability is formed. In addition, the organic semiconductor material represented by the general expression (1) has an appropriate energy level. This makes it possible to achieve favorable photoelectric conversion efficiency, superior dark current characteristics, and superior afterimage characteristics.

It is to be noted that effects described here are not necessarily limited and any of effects described in the present disclosure may be included.

### BRIEF DESCRIPTION OF DRAWINGS

FIG. 1 is a schematic cross-sectional view of a configuration of a photoelectric conversion element according to an embodiment of the present disclosure.

FIG. 2 is a schematic cross-sectional view of another example of the configuration of the photoelectric conversion element illustrated in FIG. 1.

FIG. 3 is a schematic plan view of a configuration of a unit pixel of the photoelectric conversion element illustrated in FIG. 1.

FIG. 4 is a schematic cross-sectional view for describing a method of manufacturing the photoelectric conversion element illustrated in FIG. 1.

FIG. 5 is a schematic cross-sectional view of a process following FIG. 4.

FIG. 6 is a schematic cross-sectional view of a configuration of a photoelectric conversion element according to a modification example 1 of the present disclosure.

FIG. 7 is a schematic cross-sectional view of a solar cell according to a modification example 2 of the present disclosure.

FIG. 8 is a block diagram illustrating an entire configuration of an imaging apparatus including the photoelectric conversion element illustrated in FIG. 1.

FIG. 9 is a functional block diagram illustrating an electronic apparatus (camera) rising the imaging apparatus illustrated in FIG. 8.

FIG. 10 is a block diagram depicting an example of a schematic configuration of an in-vivo information acquisition system.

FIG. 11 is a view depicting an example of a schematic configuration of an endoscopic surgery system.

FIG. 12 is a block diagram depicting an example of a functional configuration of a camera head and a camera control unit (CCU).

FIG. 13 is a block diagram depicting an example of schematic configuration of a vehicle control system.

FIG. 14 is a diagram of assistance in explaining an example of installation positions of an outside-vehicle information detecting section and an imaging section.

FIG. 15 is a schematic cross-sectional view of a photoelectric conversion element used in examples.

FIG. 16 is a characteristic diagram illustrating results of XRD measurement of an organic photoelectric conversion layer including BBBT-1 and an organic photoelectric conversion layer including BBBT-2.

FIG. 17 is a characteristic diagram illustrating results of XRD measurement of a single-layer film including BBBT-1 and a single-layer film including BBBT-2.

FIG. 18 is a diagram illustrating absorptance characteristics of BBBT-2 and BP-rBDT.

FIG. 19 is a diagram illustrating energy levels of respective organic semiconductor materials.

FIG. 20 is a characteristic diagram illustrating results of XRD measurement of an organic photoelectric conversion layer including BBBT-2 and an organic photoelectric conversion layer including BP-rBDT.

### MODES FOR CARRYING OUT THE INVENTION

In the following, some embodiments of the present disclosure are described in detail with reference to the drawings. The following description is given of specific examples of the present disclosure, and the present disclosure is not limited to the following embodiments. Moreover, the present disclosure is not limited to positions, dimensions, dimension ratios, and the like of respective components illustrated in the respective drawings. It is to be noted that description is given in the following order.
1. Embodiment (Photoelectric conversion element including an organic photoelectric conversion layer that includes a BBBT derivative represented by a general expression
1-1. Configuration of Photoelectric Conversion Element
1-2. Method of Manufacturing Photoelectric Conversion Element
1-3. Workings and Effects
2. Modification Examples
2-1. Modification Example 1 (Photoelectric conversion element in which a plurality of organic photoelectric converters are stacked)
2-2. Modification Example 2 (Solar cell)
3. Application Examples
4. Examples

### 1. EMBODIMENT'S

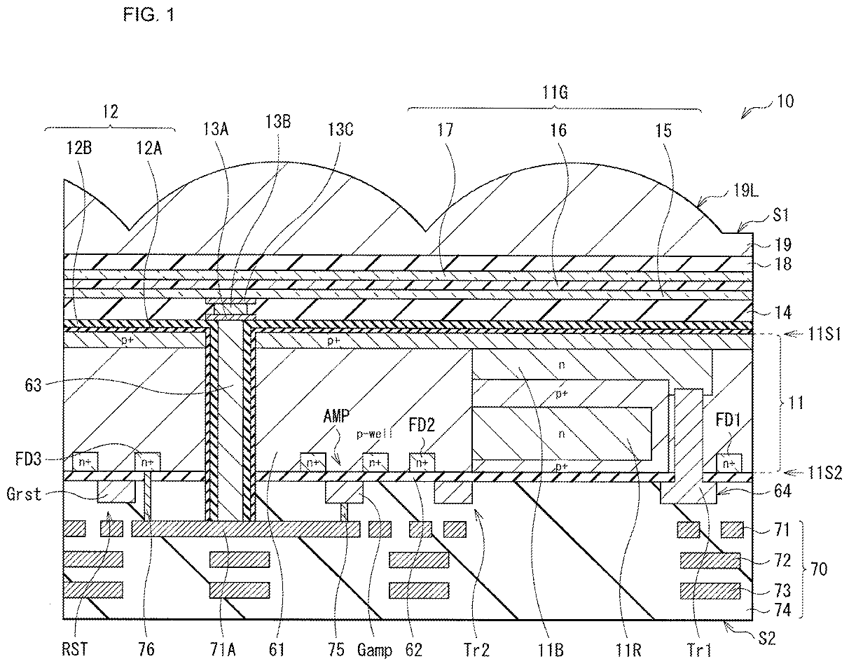

FIG. 1 illustrates a cross-sectional configuration of a photoelectric conversion element (photoelectric conversion element 10) according to an embodiment of the present disclosure. The photoelectric conversion element 10 is used, for example, as an imaging element included in one pixel (unit pixel P) of an imaging apparatus (imaging apparatus 1) such as a back-side illumination type (back-side light reception type) CCD (Charge Coupled Device) image sensor or a CMOS (Complementary Metal Oxide Semiconductor) image sensor (refer to FIG. 8). The photoelectric conversion element 10 is of a so-called longitudinal spectral type in which one organic photoelectric converter 11G and two inorganic photoelectric converters 11B and 11R are stacked in a longitudinal direction. Each of the organic photoelectric converter 11G. and the inorganic photoelectric converters 11B and 11R selectively detects light in a corresponding one of wavelength regions different from one another, and performs photoelectric conversion of the thus-detected light. In the present embodiment, an organic photoelectric conversion layer 16 included in the organic photoelectric converter 11G has a configuration formed including at least one kind of organic semiconductor material (for example, a benzobisbenzothiophene (BBBT) derivative) represented by a general expression (1) (to be described later).

### 1-1. Configuration of Photoelectric Conversion Element

The photoelectric conversion element 10 includes, in each unit pixel P, one organic photoelectric converter 11G and two inorganic photoelectric converters 11B and 11R that are stacked in the longitudinal direction. The organic photoelectric converter 11G is provided on a back surface (fist surface 11S1) of a semiconductor substrate 11. The inorganic photoelectric converters 11B and 11R are formed to be embedded in the semiconductor substrate 11. and are stacked in a thickness direction. of the semiconductor substrate 11. The organic photoelectric converter 11G includes a p-type semiconductor and an n-type semiconductor, and includes an organic photoelectric conversion layer 16 having a bulk heterojunction structure in a layer. The bulk heterojunction structure is a p-n junction surface formed by mixing the p-type semiconductor and the n-type semiconductor.

The organic photoelectric converter 11G and the inorganic photoelectric converters 11B and 11R each selectively detect light in a corresponding one of wavelength bands different from each other, and perform photoelectric conversion of the thus-detected light. Specifically, the organic photoelectric converter 11G acquires a green (G) color signal. The inorganic photoelectric converters 11B and 11R respectively acquire a blue (B) color signal and a red (R) color signal by a difference in absorption coefficient. This allows the photoelectric conversion element 10 to acquire a plurality of color signals in one pixel without using a color filter

It is to be noted that, in the present embodiment, description is given of a case where electrons of electron-hole pairs generated by photoelectric conversion are read as signal charges. Moreover, in the drawings, “+” (plus) attached to “p” or “n” indicates that p-type or n-type impurity concentration is high, and “++” indicates that p-type or n-type impurity concentration is higher than that in a case of “+”.

The semiconductor substrate 11 includes an n4ype silicon (Si) substrate, for example, and has a p-well 61 in a predetermined region. For example, various kinds of floating diffusions (floating diffusion layers) H) (for example, FD1, FD2, and FD3), various kinds of transistors Tr (for example, a vertical transistor (transfer transistor) Tr1, a transfer transistor Tr2, an amplifier transistor (modulation element) AMP, and a reset transistor RST), and multilayer wiring 70 are provided on a second surface (front surface of the semiconductor substrate 11) 11S2 of the p-well 61. The multilayer wiring 70 has, for example, a configuration in which wiring layers 71, 72, and 73 are stacked in an insulating layer 74. Moreover, a peripheral circuit (not illustrated) including a logic circuit, and the like is provided in a periphery of the semiconductor substrate 11.

It is to be noted that in FIG. 1. the first surface 11S1 side of the semiconductor substrate 11 is represented as a light incident side S1 and a second surface 11S2 side of the semiconductor substrate 11 is represented as a wiring layer side S2.

The inorganic photoelectric converters 11B and 11R each include, for example, a PIN (Positive Intrinsic Negative) photodiode, and each have a p-n junction in a predetermined region of the semiconductor substrate 11. The inorganic photoelectric converters 11B and 11R enable dispersion of light in the longitudinal direction with use of a difference in absorbed wavelength band depending on a depth of light incidence in the silicon substrate.

The inorganic photoelectric converter 11B selectively detects blue light to accumulate signal charges corresponding to blue, and is disposed at a depth that allows for efficient photoelectric conversion of blue light, The inorganic photoelectric converter 11R selectively detects red light to accumulate signal charges corresponding to red, and is disposed at a depth that allows for efficient photoelectric conversion of red light. It is to be noted that blue (B) and red (R) are colors respectively corresponding to a. wavelength band from 450 nm to 495 nm, for example, and a wavelength band from 620 nm to 750 nm, for example. It is sufficient if each of the inorganic photoelectric converters 11B and 11R is allowed to detect light in a portion. or the entirety of a corresponding one of the wavelength bands.

Specifically, as illustrated in FIG. 1, the inorganic photoelectric converter 11B and the inorganic photoelectric converter 11R each include, for example, a p+ region serving as a hole accumulation layer, and an n region serving as an electron .accumulation layer (has a p-n-p stacking structure). The n region of the inorganic photoelectric converter 11B is coupled to the vertical transistor Tr1. The p+ region of the inorganic photoelectric converter 11B bends along the vertical transistor Tr1 and is coupled to the p+ region of the inorganic photoelectric converter 11R.

For example, the floating diffusions (floating diffusion layers) FD1, FD2, and FD3, the vertical transistor (transfer transistor) Tr1, the transfer transistor Tr2, the amplifier transistor (modulation element) AMP, and the reset transistor RST are provided on the second surface 11S2 of the semiconductor substrate 11, as described above.

The vertical transistor Tr1 is a transfer transistor that transfers, to the floating diffusion FD1, signal charges (herein, electrons) corresponding to blue generated and accumulated in the inorganic photoelectric converter 11B. The inorganic photoelectric: converter 11B is formed at a position deep from the second. surface 11S2 of the semiconductor substrate 11; therefore, the transfer transistor of the inorganic photoelectric converter 11B preferably includes the vertical transistor Tr1.

The transfer transistor Tr1 transfers, to the floating diffusion FD2, signal charges (herein, electrons) corresponding to red generated and accumulated in the inorganic photoelectric converter 11R, and includes, for example, a MOS transistor.

The amplifier transistor AMP is a modulation element that modulates an amount of charges generated in the organic photoelectric converter 11G into a voltage, and includes, for example, a MOS transistor.

The reset transistor RST resets charges transferred from the organic photoelectric converter 11G to the floating diffusion FD3, and includes, for example, a MOS transistor.

A first lower contact 75, a second lower contact 76, and an upper contact 13B each include, for example, a doped silicon material such as PDAS (Phosphorus Doped Amorphous Silicon) or a metal material such as aluminum (Al), tungsten (W), titanium (Ti) , cobalt (Co), hafnium (Hf), and tantalum (Ta).

The organic photoelectric converter 11G is provided on the first surface 11S1 side of the semiconductor substrate 11. The organic photoelectric converter 11G has, for example, a configuration in which a lower electrode 15, the organic photoelectric conversion layer 16, and an upper electrode 17 are stacked in this order from the first surface 11S1 side of the semiconductor substrate 11. The lower electrode 15 is fanned separately for each photoelectric conversion element 10, for example. The organic photoelectric conversion layer 16 and the upper electrode 17 are provided as a continuous layer common to a plurality of photoelectric conversion elements 10. The organic photoelectric converter 11G is an organic photoelectric conversion element that absorbs green light corresponding to a wavelength band of a portion or the entirety of a selective wavelength band (for example, from 450 nm to 650 nm both inclusive) to generate electron-hole pairs.

For example, interlayer insulating layers 12 and 14 are stacked, between the first surface 11S1 of the semiconductor substrate 11 and the lower electrode 15, in this order from the semiconductor substrate 11 side. The interlayer insulating layer has, for example, a configuration in which a layer having fixed charges (fixed charge layer) 12A and a dielectric layer 12B having an insulation property are stacked. A protective layer 18 is provided on the upper electrode 17. An on-chip lens layer 19 is provided above the protective layer 18. The on-chip lens layer 19 includes on-chip lenses 19L and also serves as a planarization layer.

A through electrode 63 is provided between the first surface 11S1 and the second surface 11S2 of the semiconductor substrate 11. The organic photoelectric converter 11G is coupled to a gate Gamp of the amplifier transistor AMP and the floating diffusion FD3 via the through electrode 63. This allows the photoelectric conversion element 10 to well transfer charges generated in the organic photoelectric converter 11G on the first surface 11S1 side of the semiconductor substrate 11 to the second surface 11S2 side of the semiconductor substrate 11 via the through electrode 63, thereby improving characteristics.

The through electrode 63 is provided for each organic photoelectric converter 11G in each of the photoelectric conversion elements 10, for example. The through electrode 63 has a function as a connector between the organic photoelectric converter 11G and both the gate Gamp of the amplifier transistor AMP and the floating diffusion FD3, and serves as a transmission path of charges (herein, electrons) generated in the organic photoelectric converter 11G.

A lower end of the through electrode 63 is coupled to a coupling section 71A in the wiring layer 71, and the coupling section 71A and the gate Gamp of the amplifier transistor AMP are coupled to each other through a first lower contact 75, The coupling section 71A and the floating diffusion FD3 are coupled to each other through a second lower contact 76. It is to be noted that FIG. 1 illustrates the through electrode 63 having a cylindrical shape, but the through electrode 63 is not limited thereto, and may have a tapered shape, for example.

A reset gate first of the reset transistor RST is preferably disposed adjacent to the floating diffusion FD3 as illustrated in FIG. 1. This makes it possible to reset charges accumulated in the floating diffusion FD by the reset transistor RST.

In the photoelectric conversion element 10 according to the present embodiment, light having entered the organic photoelectric converter 11G from the upper electrode 17 side is absorbed by the organic photoelectric conversion layer 16. Excitons thereby generated move to an interface between an electron donor and an electron acceptor included in the photoelectric conversion layer 16, and the excitons are dissociated, that is, the excitons are dissociated into electrons and holes. Charges generated herein (electrons and holes) are each carried to different electrodes by diffusion resulting from a difference in concentration between carriers or an internal electric field resulting from a difference in work function between an anode (herein, the upper electrode 17) and a cathode (herein, the lower electrode 15), and detected as a photocurrent. Moreover, it is also possible to control transport directions of the electrons and the holes by application of a potential between the lower electrode 15 and the upper electrode 17. Herein, the anode is an electrode that receives holes, and the cathode is an electrode that receives electrons.

In the following, description is given of the configurations, materials, and the like of respective components.

The organic photoelectric converter 11G is an organic photoelectric conversion element that absorbs green light corresponding to a wavelength band of a portion or the entirety of a selective wavelength band (for example, from 450 nm to 650 nm both inclusive) to generate electron-hole pairs.

The lower electrode 15 is directly opposed to light reception surfaces of the inorganic photoelectric converters 11B and 11R formed in the semiconductor substrate 11, and is provided in a region covering these light reception surfaces. The lower electrode 15 includes an electrically conductive film having light transmissivity, and includes, for example, a metal oxide having electrical conductivity. Specifically, the lower electrode 15 includes a transparent electrically conductive material such as indium oxide (In₂O₃), tin-doped In₂O₃ (ITO), indium-tin oxide (ITO) including crystalline ITO and amorphous ITO, indium-zinc oxide (IZO) prepared by adding indium as a dopant to zinc oxide, indium-gallium oxide (IGO) prepared by adding indium as a dopant to gallium oxide, indium-gallium-zinc oxide (IGZO, In—GaZnO₄) prepared by adding indium and gallium as dopants to zinc oxide, IFO (F-doped In₂O₃), tin oxide (SnO₂), ATO (Sb-doped SnO₂). FTO (F-doped SnO₂), zinc oxide (including ZnO doped with any other element), aluminum-zinc oxide (AZO) prepared by adding aluminum as a dopant to zinc oxide, galliumzinc oxide (GZO) prepared by adding gallium as a dopant to zinc oxide, titanium oxide (TiO₂), antimony oxide, a spinel oxide, and an oxide having a YbFe₂O₄ structure. Other than these materials, the lower electrode 15 may have a transparent electrode Structure including gallium oxide, titanium oxide, niobium oxide, nickel oxide, or the like as a base layer A thickness of the lower electrode 15 is, for example, from 20 nm to 200 nm both inclusive, and preferably from 30 nm to 100 nm both inclusive.

The photoelectric conversion layer 16 converts optical energy into electric energy. The photoelectric conversion layer 16 includes one or more kinds of organic semiconductor materials, and preferably includes one or both of a p-type semiconductor and an n-type semiconductor, for example. For example, in a case where the organic photoelectric conversion layer 16 includes two kinds of organic semiconductor materials, that is, the p-type semiconductor and the n-type semiconductor; one of the p-type semiconductor and the n-type semiconductor is preferably a material having transmissivity to visible light, and the other is preferably material that performs photoelectric conversion of light in a selective wavelength region (for example, from 450 nm to 650 nm both inclusive). Alternatively, the organic photoelectric conversion layer 16 preferably includes three kinds of organic semiconductor materials, that is, a material (light absorber) that performs photoelectric conversion of light in a selective wavelength region and an n-type semiconductor and a p-type semiconductor that have transmissivity to visible light. In the present embodiment, as the p-type semiconductor, at least one kind of organic semiconductor material represented by the following general expression (1) is included.

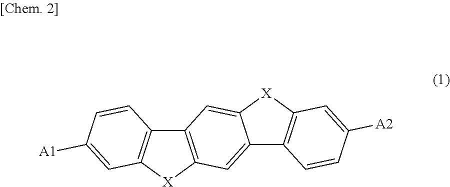

(X is one of an oxygen atom (O), a sulfur Atom (S), and a selenium atom (Se), and A1 and A2 are each independently an aryl group, a heteroary group, an aryl amino group, a heteroaryl amino group, an aryl group having an aryl amino group as a substituent group, an aryl group having a heteroaryl amino group as a substituent group, a heteroaryl group having an aryl amino group as a substituent group, a heteroaryl group having a heteroaryl amino group as a substituent group, or a derivative thereof.)

Aryl substituent groups of the above-described aryl group and the above-described above aryl amino group include a phenyl group, a biphenyl phenyl group, a naphthyl group, a naphthylphenyl group, a naphthylbiphenyl group, a phenylnaphthyl group, a tolyl group a xylyl group, a terphenyl group, an anthracenyl group, a phenanthryl group, a pyrenyl group, a tetracenyl group, and a fluoranthenyl group. Heteroaryl substituent groups of the above-described heteroaryl group and the above-described heteroaryl amino group include a thienyl group, a thienyl phenyl group, a thienyl biphenyl group, a thiazolyl group, a thiazolyl phenyl group, a thiazolyl biphenyl group, an isothiazolyl group, an isothiazolyl phenyl group, an isothiazolyl biphenyl group, a furanyl group, a furanyl phenyl group, a furanyl biphenyl group, an oxazolyl group, an oxazolyl phenyl group, an oxazolyl biphenyl group, an oxadiazolyl group, an oxadiazolyl phenyl group, an oxadiazolyl biphenyl group, an isooxazolyl group, a benzothienyl group, a benzothienyl phenyl group a benzothienyl biphenyl group, a beuzofuranyl group, a pyridinyl group, a pyridinyl phenyl group, a pyridinyl biphenyl group, a quinolinyl group, a quinolyl phenyl group a quinolyl biphenyl group, an isoquinolyl group, arr isoquinolyl phenyl group, an isoquinolyl biphenyl group, an acridinyl group, an indole group, an indole phenyl group, an indole biphenyl group, an imidazole group, an imidazole phenyl group, an imidazole biphenyl group, a benzimidazole group, a benzimidazole phenyl group, a benzimidazole biphenyl gray, and a carbazolyl group.

The organic semiconductor material represented by the above-described general expression (1) preferably has transmissivity to visible light, for example. Specifically, the organic semiconductor material in a single-layer film having a film thickness of S ruin to 100 nm both inclusive preferably has a light absorptance of 0% to 3% both inclusive at a wavelength of 450 nm or greater, a light absorptance of 0% to 30% both inclusive at a wavelength of 425 nm and a light absorptance of 0% to 80% both inclusive at a wavelength of 400 nm, In addition, the organic semiconductor material represented by the above-described general expression (1) preferably has an energy difference of 1.1 eV or greater between an apparent HOMO level in the organic photoelectric conversion layer 16 and a LUMO level of a material other than the organic semiconductor material represented by the general expression (1) in the organic photoelectric conversion layer. Here, the apparent HOMO level is obtained by measuring an ionization potential represented by the organic semiconductor material in the general expression (1) inside the photoelectric conversion layer with use of a GCIB-UPS apparatus having a combination of ultraviolet photoelectron spectroscopy (UPS) and a gas cluster ion gun (GCIB) in a case where a material other than the organic semiconductor material represented by the general expression (1) is also included in the photoelectric conversion layer.

Examples of the organic semiconductor material represented by the above-described general expression (1) include a benzobisbenzothiophene (BBBT) derivative represented by the following general expression (1′). Specific examples thereof include compounds represented by the following expressions (1-1) and (1-2).

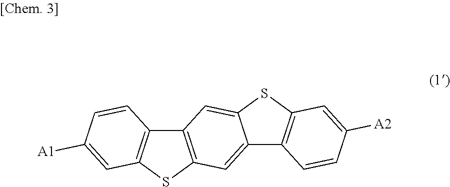

(A1 and A2 are each independently an aryl group, a heteroaryl group, an aryl amino group, a heteroaryl amino group, an aryl group having an aryl amino group as a substituent group, an aryl group having a heteroaryl amino group as a substituent group, a heteroaryl group having an aryl amino group as a substituent group, a heteroaryl group having a heteroaryl amino group as a substituent group, or a derivative thereof.)

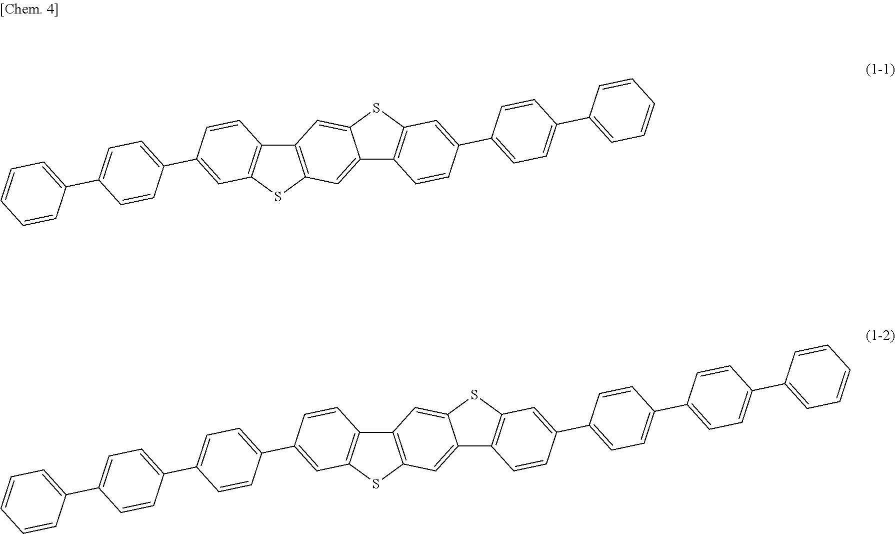 [SMILES: c1ccc(-c2ccc(-c3ccc4c(c3)sc3cc5c(cc34)sc3cc(-c4ccc(-c6ccccc6)cc4)ccc35)cc2)cc1 ; c1ccc(-c2ccc(-c3ccc(-c4ccc5c(c4)sc4cc6c(cc45)sc4cc(-c5ccc(-c7ccc(-c8ccccc8)cc7)cc5)ccc46)cc3)cc2)cc1]

The organic photoelectric conversion layer 16 preferably uses, for example, fullerene C60 represented by the following general expression (2) or a derivative thereof, or fullerene C70 represented by the following general expression (3) or a derivative thereof, in addition to the above-described BBBT derivative. Using at least one kind of fullerene C60, fullerene C70, and derivatives thereof makes it possible to further improve photoelectric conversion efficiency.

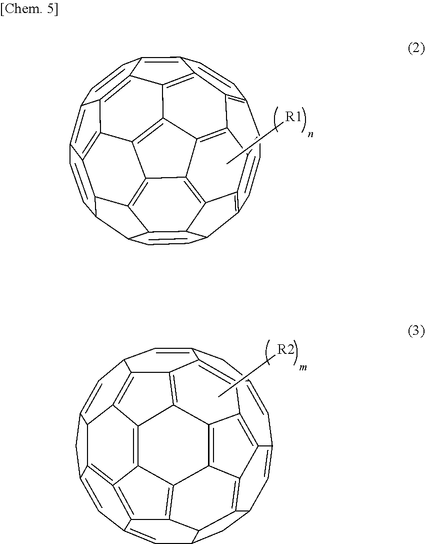

(R1 and R2 are each a hydrogen atom, a halogen atom, a straight-chain, branched, or cyclic alkyl group, a phenyl group, a group having a straight-chain or condensed ring aromatic compound, a group having a halogen compound, a partial fluoroalkyl group, a perfluoroalkyl group, a silyl alkyl group, a silyl alkoxy group, an aryl silyl group, an aryl sulfanyl group, an alkyl sulanyl group, an aryl sulfonyl group, an alkyl sulfonyl group, an aryl sulfide group, an alkyl sulfide group, an amino group, an alkyl amino group, an aryl amino group a hydroxy group, an alkoxy group, an acyl amino group, an acyloxy group, a carbonyl group, a carboxy group, a carboxamide group, a carboalkoxy group, an acyl group, a sulfonyl group, a cyano group, a nitro group, a group having a chalcogen compound, a phosphine group, a phosphone group, or any of derivatives thereof. Each of n and m is 0 or an integer of 1 or greater.)

The organic photoelectric conversion layer 16 preferably uses a material (light absorber) that performs photoelectric conversion of light in a selective wavelength region, in addition to the above-described BBBT derivative. For example, an organic semiconductor material having an absorption maximum wavelength at a wavelength longer than blue light (a wavelength of 450 nm) is preferably used, and more specifically, for example, an organic semiconductor material having an absorption maximum wavelength in a wavelength region from 500 nm to 600 nm both inclusive is preferably used. This makes it possible to selectively perform photoelectric conversion of green light in the organic photoelectric converter 11G. Examples of such materials include subphthalocyanine represented by the following general expression (4) and a derivative thereof.

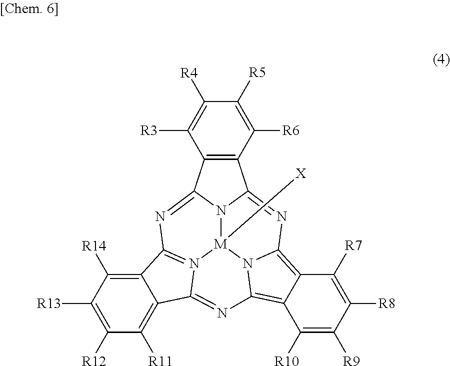

(R3 to R14 are each independently selected from a group configured of a hydrogen atom, a halogen atom, a straight-chain, branched, or cyclic alkyl group, a thioalkyl group, a thioaryl group, an aryl sulfonyl group, an alkyl sulfonyl group, an amino group, an alkyl amino group, an aryl amino group, a hydroxy group, an alkoxy group, an acyl amino group, an acyloxy group, a phenyl group, a carboxy group, a carboxamide group, a carboalkoxy group, an acyl group, a sulfonyl group, a cyano group, and a nitro group, and any adjacent ones of R3 to R14 are optionally part of a condensed aliphatic ring or a condensed aromatic ring. The condensed aliphatic ring or the condensed aromatic ring described above optionally includes one or a plurality of atoms other than carbon. M is boron or a divalent or trivalent metal. X is a substituent group of one selected from a group configured of a halogen, a hydroxy group, a thiol group, an imide group, a substituted or unsubstituted alkoxy group, a substituted or unsubstituted aryloxy group, a substituted or unsubstituted alkyl group, a substituted or unsubstituted alkythio group, and a substituted or unsubstituted arylthio group.)

The organic photoelectric conversion layer 16 is preferably formed using one kind of the above-described BBBT derivative, one kind of subphthalocyanine or a derivative thereof and one kind of fullerene C60, fullerene C70, or a derivative thereof. A combination of the above-described BBBT derivative, subphthalocyanine or the derivative thereof, and fullerene C60, fullerene C70, or a derivative thereof function as a p-type semiconductor or an n-type semiconductor depending on materials to be combined together.

In addition, the organic photoelectric conversion layer 16 may include the following organic semiconductor materials as a p-type semiconductor and an n-type semiconductor in addition to the above-described materials.

Examples of the p-type semiconductor include a naphthalene derivative, an anthracene derivative, a phenanthrene derivative, a pyrene derivative, a perylene derivative, a tetracene derivative, a pentacene derivative, and a quinacridone derivative. Further, the examples include thienoacene-based materials typified by a thiophene derivative, a thienothiophene derivative, a benzothiophene derivative, a benzothienobenzothiophene (BTBT) derivative, a dinaphthothienothiophene (DNTT) derivative, a dianthracenothienothiophene (DATT) derivative, thienobisbenzothiophene (TBBT) derivative, a dibenzothienobisbenzothiophene (DBTBT) derivative, a dithienobenzodithiophene (DTBDT) derivative, a dibenzothienothiophene (DBTDT) derivative, a benzodithiophene (BDT) derivative, a naphthodithiophene (NDT) derivative, an anthracenodithiophene (ADT) derivative, a tetracenodithiophene (TDT) derivative, and a pentacenodithiophene (PDT) derivative. In addition to these materials, the examples include a triallylamine derivative, a carbazole derivative, a picene derivative, a chrysene derivative, a fluoranthene derivative, a plithalocyanine derivative, a subphthalocyanine derivative, subporphyrazine derivative, a metal complex having a heterocyclic compound as a ligand, a polythiophene derivative, a polybenzothiadiazole derivative, a polyfluorene derivative, and the like.

Examples of the n-type semiconductor include higher fullerenes such as fullerene C74, endohedral fullerenes, and derivatives thereof (for example, a fullerene fluoride, a PCBM fullerene compound, a fullerene multimer, and the like), in addition to fullerene C60 and fullerene C70. In addition to these materials, it is possible to use a an organic semiconductor having a lager HOMO value and larger LUMO (Lowest Unoccupied Molecular Orbital) value than the p-type semiconductor, a transparent inorganic metal oxide. Specific examples thereof include a heterocyclic compound including a nitrogen atom, an oxygen atom, a sulfur atom. Examples of the heterocyclic compound include a pyridine derivative, a pyrazine derivative, a pyrimidine derivative, a triazine derivative, a quinoline derivative, a quinoxaline derivative, an isoquinoline derivative, an acridine derivative, a phenazine derivative, a phenanthroline derivative, a tetrazole derivative, a pyrazole derivative, an imidazole derivative, a thiazole derivative, an oxazole derivative, an imidazole derivative, a benzimidazole derivative, a benzotriazole derivative, a benzoxazole derivative, a benzoxazole derivative carbazole derivative, a benzofuran derivative, a dibenzofuran derivative, a subporphyrazine derivative, a polyphenylene vinylene derivative, a polybenzothiadiazole derivative, an organic molecule having a polyfluorene derivative or the like in a portion of a molecular skeleton. an organic metal complex, and a subphthalocyanine derivative. Examples of a group or the like included in a fullerene derivative include a halogen atom, a straight-chain, branched, or cyclic alkyl group or phenyl group, a group having a straight-chain or condensed aromatic compound, a group having a halide, a partial fluoroalkyl group a perfluoroalkyl group, a silyl alkyl group, a silyl alkoxy group, an aryl silyl group, an aryl sulfanyl group, an alkyl sulfanyl group, an aryl sulfonyl group, an alkyl sulfonyl group, an aryl sulfide group, an alkyl sulfide group, an amino group, an alkyl amino group, an aryl amino group, a hydroxy group, an alkoxy group, an acyl amino group, an acyloxy group, a carbonyl group, a carboxy group, a carboxamide group, a carboalkoxy group, an acyl group, a sulfonyl group, a cyano group, a nitro group, a group having a chalcogenide, a phosphine group, a phosphone group, and derivatives thereof.

The organic photoelectric conversion layer 16 may have a single-layer structure or a stacked structure. In a case where the organic photoelectric conversion layer 16 is configured as a single-layer structure, as described above, for example, it is possible to use one or both of the p-type semiconductor and the n-type semiconductor. In a case where the organic photoelectric conversion layer 16 is configured with use of both the p-type semiconductor and the n-type semiconductor, the p-type semiconductor and the n-type semiconductor are mixed to form a bulk heterostructure in the organic photoelectric conversion layer 16. In this organic photoelectric conversion layer 16, a material (light absorber) that performs photoelectric conversion of light in a selective wavelength region may be further fixed. In a case where the organic photoelectric conversion layer 16 is configured as a stacked structure, examples of the stacked structure include two-layer structures of the p-type semiconductor layer/the n-type semiconductor layer, the p-type semiconductor layer/a mixed layer (bulk heterolayer) including the p-type semiconductor and the n-type semiconductor, and the n-type semiconductor layer/a mixed layer (bulk heterolayer) including the p-type semiconductor and the n-type semiconductor, or a three-layer structure of the p-type semiconductor layer/a mixed layer (bulk heterolayer) including the p-type semiconductor and the n-type semiconductor/the n-type semiconductor layer. It is to be noted that respective layers included in the organic photoelectric conversion layer 16 may include two or more kinds of p-type semiconductors and two or more kinds of n-type semiconductors.

The thickness of the organic photoelectric conversion layer 16 is not specifically limited, but the thickness may be, for example, from 10 nm to 500 nm both. inclusive, preferably from 25 nm to 300 nm both inclusive, more preferably from 25 nm to 200 nm both inclusive, and still more preferably from 100 nm to 180 nm both inclusive.

It is to he noted that organic semiconductors are often classified into a p type and an n type, but the p type means that holes are easily transported, and the n type means that electrons are easily transported. The p-type and the n-type in organic semiconductors are not limited to an interpretation that the organic semiconductor has holes or electrons as many carriers of thermal excitation similarly to an inorganic semiconductor.

The upper electrode 17 includes an electrically conductive film having light transmissivity similarly to the lower electrode 15. In the imaging apparatus 1 using the photoelectric conversion element 10 as one pixel, the upper electrode 17 may be separately provided for each of the pixels, or may be formed as a common electrode for the respective pixels. A thickness of the upper electrode 17 is, for example, from 10 nm to 200 nm both inclusive, and preferably from 30 nm to 100 nm both inclusive.

Further, the lower electrode 15 and the upper electrode 17 may be covered with an insulating material. Examples of a material of a coating layer that covers the lower electrode 15 and the upper electrode 17 include inorganic insulating materials forming a high dielectric insulating film, such as a silicon oxide-based material and a metal oxide such as silicon nitride (SiNₓ) and aluminum oxide (Al₂O₃). In addition, polymethyl metacrylate (PMMA), polyvinyl phenol (PVP), polyvinyl alcohol (PVA), polyimide polycarbonate (PC), polyethylene terephthalate (PET), polystyrene, a derivative coupling agent) such as N-2 (aminoethyl)3-aminopropyltrimethoxysilane (AEAPTMS), 3-mercaptopropyltrimethoxysilane (MPTMS), and octadecyltrichlorosilane (OTS), or an organic insulating material (organic polymer) such as straight-chain hydrocarbons having a functional group that is able to be bonded to an electrode at one end of octadecanethiol, dodecyl isocyanate, or the like may be used. In addition, a combination of these materials may be used. It is possible to use a combination of these materials. It is to be noted that examples of the silicon oxide-based material include silicon oxide (SiOx), BPSG, PSG, BSG, AsSG, PbSG, silicon oxynitride (SiON), SOG (spin-on glass), a low dielectric material (for example, polyarylether cycloperfluorocarbon polymer, benzocyclobutene, a cyclic fluorine resin, polytetratluoroethylene, fluorinated aryl ether, fluorinated polyimide, amorphous carbon, and organic SOG). As a method of forming the coating layer, for example, it is possible to use a dry film formation method and a wet film formation method that are to be described later.

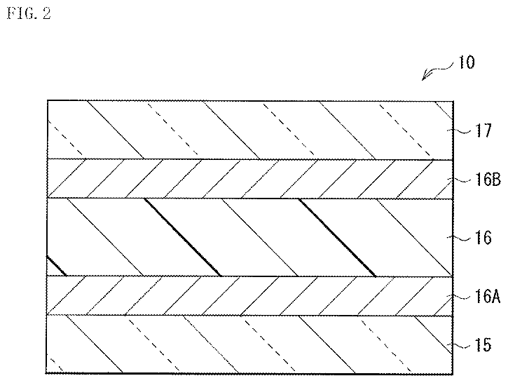

It is to he noted that any other layer may be provided between the organic photoelectric conversion layer 16 and the lower electrode 15 and between the organic photoelectric conversion layer 16 and the upper electrode 17. Specifically, for example, as illustrated in FIG. 2, buffer layers 16A and 16B may be provided respectively between the organic photoelectric conversion layer 16 and the lower electrode 15 and between the organic photoelectric conversion layer 16 and the upper electrode 17.

The buffer layer 16A improves electrical bondability between the organic photoelectric conversion layer 16 anal the lower electrode 15. In addition, the buffer layer 16A serves to adjust electrical capacitance of the photoelectric conversion element 10. As a material of the buffer layer 16A, as with the following buffer layer 16B, it is possible to use the organic semiconductor material represented by the above-described general expression (1) such as a BBBT derivative. Other than this material, a material having a larger (deeper) work function than a material used in the buffer layer 16B is preferably used. Specifically, a preferable example is a material that is an organic molecule and an organic metal complex having, as a portion of a molecular skeleton, a heterocyclic ring including nitrogen (N) such as pyridine, quinoline, acridine, indole, imidazole, benzimidazole phenanthroline, naphthalenetetracarboxdiimide, naphthalene dicarboxylic acid monoimide, hexaazatriphenylene, and hexaazatrinaphtylene, and has small absorption in a visible region. In addition, in a case where the buffer layer 16A that is a thin film having a thickness of about 5 nm to about 20 nm is used as a charge blocking layer on a cathode side, it is possible to use a fullerene typified by fullerene C60 and fullerene C70 having absorption in a visible light region from 400 nm to 700 nm and a derivative thereof.

The buffer layer 16B improves electrical bondability between the upper electrode 17 and the organic photoelectric conversion layer 16. In addition, the buffer layer 16B serves to adjust electrical capacitance of the photoelectric conversion element 10. As a material of the buffer layer 16B, the organic semiconductor material represented by the above-described general expression (1) such as a BBBT derivative is preferably used. In addition to the organic semiconductor material, aromatic amine-based materials typified by a triallylamine compound, a benzidine compound, and a styrylamine compound, a carbazole derivative, an indolocarbazole derivative, a naphthalene derivative, an anthracene derivative, a phenanthrene derivative, a pyrene derivative, a perylene derivative, a tetracene derivative, a pentacene derivative, a perylene derivative, a picene derivative, a chrysene derivative, a fluoranthen derivative, a phthalocyanine derivative, a subphthalocyanine derivative, a hexaazatriphenylene derivative, and a metal complex including a heterocyclic compound as a ligand are used. In addition, thienoacene-based materials typified by a thiophene derivative, a thienothiophene derivative, a beuzothiopherre derivative, a benzothienobenzothiophene (BTBT) derivative, a dinaphthothienothiophene (DNTT) derivative, a dianthracenothienothiophene (DATT) derivative, thienobisbenzothiophene (TBBT) derivative, a dibenzothienobisbenzothiophene (DBTBT) derivative, a dithienobenzodithiophene (DTBDT) derivative, a dibenzothienothiophene (DBTDT) derivative, a benzodithiophene (BDT) derivative, a naphthodithiophene (NDT) derivative, an anthracenodithiophene (ADT) derivative, a tetracenodithiophene (TDT) derivative and a pentacenodithiophene (PDT) derivative are used. Further, compounds such as poly(3,4-ethylenedioxythiophene)/polystyrene sulfonate [PEDOT/PSS] polyaniline, molybdenum oxide (MoOx), ruthenium oxide (RuOx), vanadium Oxide (VOx), and tungsten oxide (WOx) are used. In particular, in a case where a film thickness of the buffer layer 16B is increased in order to greatly reduce electrical capacitance, a thienoacene-based material having high carrier transportability is preferably used.

It is to be noted that the buffer layers 16A and 16B may have a single-layer structure or a stacked structure, as with the organic photoelectric conversion layer 16. A thickness per layer of the buffer layers 16A and 16B is not specifically limited, but may be, for example, from 5 nm to 500 nm both inclusive, preferably from 5 nm to 200 nm both inclusive, and more preferably from 5 nm to 100 nm both inclusive. In addition, for example, an undercoat film, a hole transport layer, an electron blocking film, the organic photoelectric conversion layer 16, a hole blocking layer, an electron transport layer, a work function adjustment film, and the like may be stacked in order from the upper electrode 17.

The fixed charge layer 12A may be a film having positive fixed charges or a film having negative fixed charges. Examples of a material of the film having the negative fixed charges include hafnium oxide, aluminum oxide, zirconium oxide, tantalum oxide, titanium oxide, and the like. In addition, as a material other than the above-described materials, lanthanum oxide, praseodymium oxide, cerium oxide, neodymium oxide, promethium oxide, samarium oxide, europium oxide, gadolinium oxide, terbium oxide, dysprosium oxide, holmium oxide, thulium oxide, ytterbium oxide, lutetium oxide, yttrium oxide, an aluminum nitride film, a hafnium oxynitride film, an aluminum oxynitride film, or the like may also be used.

The fixed charge layer 12A may have a configuration in which two or more kinds of films are stacked. This makes it possible to further enhance a function as a hole accumulation layer, for example, in the case of the film having the negative fixed charges.

Although a material of the dielectric layer 12B is not specifically limited, the dielectric layer 12B is formed using, for example, a silicon oxide film, TEOS, a silicon nitride film, a silicon oxynitride film, or the like.

The interlayer insulation layer 14 includes, for example, a single-layer film including one kind of silicon oxide, silicon nitride, silicon oxynitride (SiON), and the like, or a stacked film including two or more kinds thereof.

The protective layer 18 includes a material having light transmissivity, and includes, for example, a single-layer film including, one of silicon oxide, silicon nitride, silicon oxynitride, and the like, or a stacked film including two or more kinds thereof. A thickness of the protective layer 18 is, for example, from 100 nm to 30000 nm.

The on-chip lens layer 19 is formed on the protective layer 18 to cover an entire surface of the protective layer 18. A plurality of on-chip lenses 19L (microlenses) is provided on a front surface of the on-chip lens layer 19. The on-chip lenses 19L concentrates light incoming from above the on-chip lenses 19L onto each of light reception surfaces of the organic photoelectric converter 11G and the inorganic photoelectric converters 11B and 11R. In the present embodiment, the multilayer wiring 70 is formed on the second surface 11S2 side of the semiconductor substrate 11, which makes it possible to dispose the respective light reception surfaces of the organic photoelectric converter 11G and the inorganic photoelectric converters 11B and 11R close to one another, and to reduce variation in sensitivity between respective colors that is caused depending on an F-number of the on-chip lenses 19L.

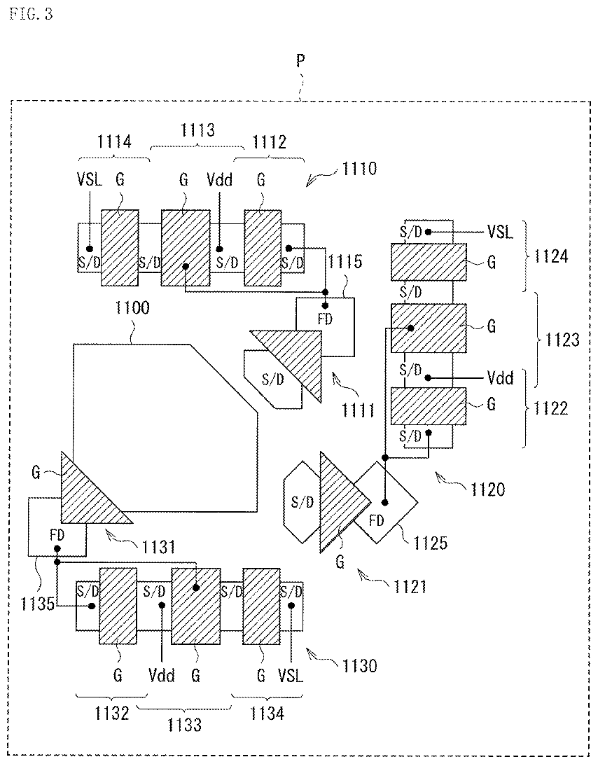

FIG. 3 is a plan view of a configuration example of an imaging element including a pixel in which a plurality of photoelectric converters (for example, the inorganic photoelectric converters 11B and 11R and the organic photoelectric converter 11G described above) to which the technology according to the present disclosure is applicable are stacked. That is, FIG. 2 illustrates an example of a planar configuration of the unit pixel P included in a pixel section la illustrated in FIG. 8.

The unit pixel P includes a photoelectric conversion region 1100 in which a red photoelectric converter (the inorganic photoelectric converter 11R in FIG. 1), a blue photoelectric converter (the inorganic photoelectric converter 11B in FIG. 1), and a green photoelectric converter (the organic photoelectric converter 11G in FIG. 1) that respectively perform photoelectric conversion of light of wavelengths of R (Red), G (Green), and B (Blue) (any of them is not illustrated in FIG. 3) are stacked in three layers in order of the green photoelectric converter, the blue photoelectric converter, and the red photoelectric converter from a light reception surface (the light incident side S1 in FIG. 1), for example. Further, the unit pixel P includes a Tr group 1110, a Tr group 1120, and a group 1130 as charge readout sections that respectively read charges corresponding to light of wavelengths of R, G, and B from the red photoelectric converter, the green photoelectric converter, and the blue photoelectric converter. In the imaging apparatus 1, in one unit pixel, dispersion in the longitudinal direction that is, dispersion of light of RGB is respectively performed in the layers as the red photoelectric converter, the green photoelectric converter, and the blue photoelectric converter stacked in the photoelectric conversion region 1100.

The Tr group 1110, the Tr group 1120, and the Tr group 1130 are formed on the periphery of the photoelectric conversion region 1100. The Tr group 1110 outputs, as a pixel signal, signal charges corresponding to light of R generated and accumulated in the red photoelectric converter. The Tr group 1110 includes a transfer Tr (MOS FET) 1111, a reset Tr 1112, an amplification Tr 1113, and a selection Tr 1114. The Tr group 1120 outputs, as a pixel signal, signal charges corresponding to light of B generated and accumulated in the blue photoelectric converter. The Tr group 1120 includes a transfer Tr 1121, a reset Tr 1122, an amplification Tr 1123, and a selection Tr 1124. The Tr group 1130 outputs, as a pixel signal, signal charges corresponding to light of G generated and accumulated in the green photoelectric converter. The Tr group 1130 includes a transfer Tr 1131, a reset Tr 1132, an amplification Tr 1133, and a selection Tr 1134.

The transfer Tr 1111 includes a gate G, a source/drain region S/D, and an FD (floating diffusion) 1115 (source/drain region serving as the FD 1115). The transfer Tr 1121 includes the gate G, the source/drain region S/D and an FD 1125. The transfer Tr 1131 includes the gate G, the peen photoelectric converter (that is, the source/drain region S/D coupled to the green photoelectric converter) in the photoelectric conversion region 1100, and an FD 1135. It is to be noted that the source/drain region of the transfer Tr 1111 is coupled to the red photoelectric converter in the photoelectric conversion region 1100, and the source/drain region S/D of the transfer Tr 1121 is coupled to the blue photoelectric converter in the photoelectric conversion region 1100.

Each of the reset Trs 1112, 1132, and 1122, the amplification Trs 1113, 1133, and 1123, and the selection Trs 1114, 1134, and 1124 includes the gate G and a pair of source/drain regions S/D that are disposed to interpose the gate G therebetween,

The FDs 1115, 1135, and 1125 are respectively coupled to the source/drain regions S/D serving as sources of the reset Trs 1112, 1132, and 1122, and are respectively coupled to the gates G of the amplification Trs 1113, 1133, and 1123. A power source Vdd is coupled to each of the source/drain region S/D common to the reset Tr 1112 and the amplification Tr 1113, the source/drain region S/D common to the reset Tr 1132 and the amplification Tr 1133, and the source/drain region S/D common to the reset Tr 1122 and the amplification Tr 1123. A VSL (vertical signal line) is coupled to each of the source/drain regions S/D serving as sources of the selection Trs 1114, 1134, and 1124.

The technology according to the present disclosure is applicable to the imaging element described above.

### 1-2. Method of Manufacturing Photoelectric Conversion Element

It is possible to manufacture the photoelectric conversion element 10 according to the present embodiment in the following manner, for example.

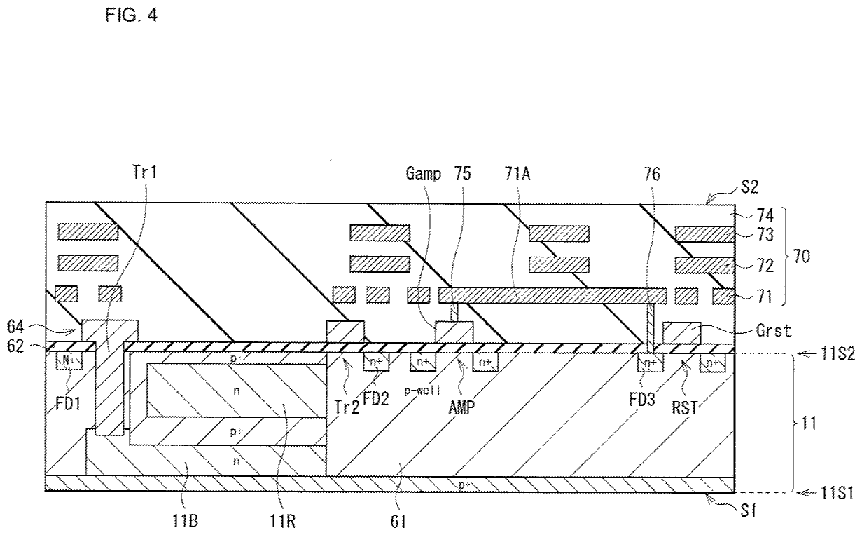

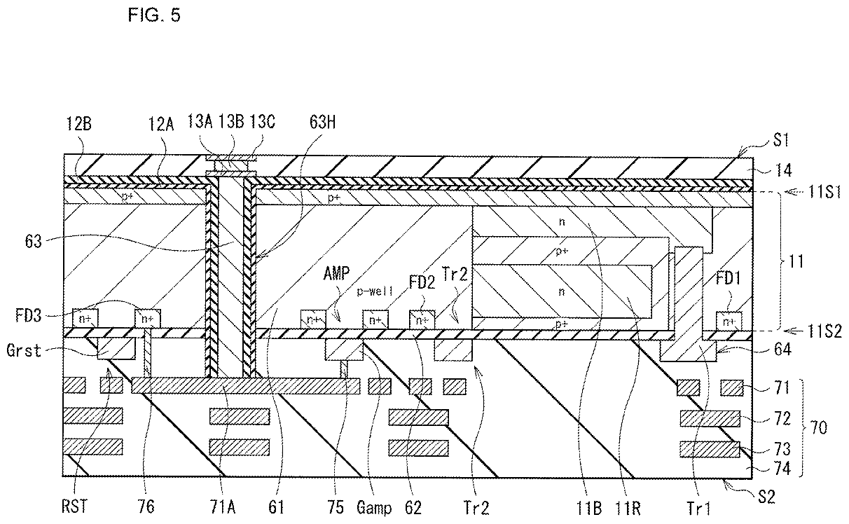

FIGS. 4 and 5 illustrate a method of manufacturing the photoelectric conversion element 10 in process order. First, as illustrated in FIG. 4, for example, the p-well 61 is formed as a well of a first conductivity type in the semiconductor substrate 11, and the inorganic photoelectric converters 11B and 11R of a second conductivity type (for example, the n type) are formed in this p-well 61. A p+ region is formed in the vicinity of the first surface 11S1 of the semiconductor substrate 11.

Similarly, as illustrated in FIG. 4, on the second surface 11S2 of the semiconductor substrate 11, n+ regions serving as the floating diffusions FD1 to FD3 are formed, and thereafter, a gate wiring layer 62, and a gate wiring layer 64 including respective gates of the vertical transistor Tr1, the transfer transistor Tr2, the amplifier. transistor AMP, and the reset transistor RST are formed. Thus, the vertical transistor Tr1, the transfer transistor Tr2, the amplifier transistor AMP, and the reset transistor RST are formed. Furthermore, the multilayer wiling 70 including the first lower contact 75, the second lower contact 76, the wiring layers 71 to 73 including the coupling section 71A, and the insulation layer 74 is formed on the second surface 11S2 of the semiconductor substrate 11.

As a base substrate of the semiconductor substrate 11 an SOI (Silicon on Insulator) substrate in which the semiconductor substrate 11, an embedded oxide film (not illustrated), and a retaining substrate (not illustrated) are stacked is used. The embedded oxide film and the retaining substrate are not illustrated in FIG. 4, but are joined to the first surface 11S1 of the semiconductor substrate 11. Annealing treatment is performed after ion implantation.

Next, a supporting substrate (not illustrated), another semiconductor substrate, or the like is joined to the second surface 11S2 side of the semiconductor substrate 11 (on the multilayer wiring 70 side) and flipped from top to bottom. Subsequently, the semiconductor substrate 11 is separated from the embedded oxide film and the retaining substrate of the SOI substrate to cause the first surface 11S1 of the semiconductor substrate 11 to be exposed. It is possible to perform the above processes with technologies used in a typical CMOS process such as ion implantation and CVD (Chemical Vapor Deposition).

Next, as illustrated in FIG. 5, the semiconductor substrate 11 is processed from the first surface 11S1 side by dry etching, for example, to form an annular opening 63H. As illustrated in FIG. 5, a depth of the opening 63H preferably penetrates from the first surface 11S1 to the second surface 11S2 of the semiconductor substrate 11 and reaches the coupling section 71A, for example.

Subsequently, as illustrated in FIG. 5, for example, the negative fixed charge layer 12A is formed on the .first surface 11S1 of the semiconductor substrate 11 and a side surface of the opening 63H. Two or more kinds of films may be stacked as the negative fixed charge layer 12A. This makes it possible to further enhance a function as the hole accumulation layer. After the negative fixed charge layer 12A is formed, the dielectric layer 12B is formed.

Subsequently, the opening 63H is filled with an electrical conductor to form the through electrode 63. As the electrical conductor, other than a doped silicon material such as PDAS (Phosphorus Doped Amorphous Silicon), it is possible to use a metal material such as aluminum (Al), tungsten (W), titanium (Ti), cobalt (Co), hafnium (Hf), and tantalum (Ta).

Subsequently, a pad section 13A is formed on the through electrode 63, and thereafter, the interlayer insulating layer 14 is formed on the dielectric layer 12B and the pad section 13A. In the interlayer insulating layer 14, the upper contact 13B and a pad section 13C that electrically couple the lower electrode 15 and the through electrode 63 (specifically the pad section 13A on the through electrode 63) to each other are provided on the pad section 13A.

Subsequently, the lower electrode 15, organic layers such as the organic photoelectric conversion layer 16, the upper electrode 17, and the protective layer 18 are formed in this order on the interlayer insulating layer 14. As a method of forming films of the lower electrode 15 and the upper electrode 17, it is possible to use a dry method or a wet method. The dry method includes a physical vapor deposition method (PVD method) and a chemical vapor deposition method (CVD method). Film formation methods using the principle of the PVD method include a vacuum evaporation method using resistance heating or high-frequency heating, an EB (electron beam) evaporation method, various kinds of sputtering methods (a magnetron sputtering method, an RF-DC coupled bias sputtering method, an ECR sputtering method, a facing-target sputtering method and a high frequency sputtering method), an ion plating method, a laser ablation method, a molecular beam epitaxy method, and a laser transfer method. As the CVD method, it is possible to use a plasma CVD method, a thermal CVD method, an organic metal (MO) CVD method, and a photo CVD method. In contrast, the wet method includes an electroplating method, an electroless plating method, a spin coating method, an inkjet method, a spray coating method, a stamp method, a microcontact printing method, a flexographic printing method, an offset printing method, a gravure printing method, a dipping method, and the like. For patterning, it is possible to use chemical etching such as shadow mask, laser transfer, and photolithography, and physical etching by ultraviolet light, laser, or the like, and the like. As a planarization technology, it is possible to use a laser planarization method, a reflow method, a chemical mechanical polishing method (CMP method), and the like.

As a method of forming films of various organic layers (for example, the organic photoelectric conversion layer 16 and the buffer layers 16A and 16B), a dry film formation method and a wet film formation method are used as with the lower electrode 15 and the upper electrode 17. The dry film formation method include a vacuum evaporation method using resistance heating or high-frequency heating, an EB (electron beam) evaporation method, various kinds of sputtering methods (a magnetron sputtering method, an RF-DC coupled bias sputtering method, an ECR sputtering method, a facing-target sputtering method and a high frequency sputtering method), an ion plating method, a laser ablation method, a molecular beat epitaxy method, and a laser transfer method. As the CVD method, it is possible to use a plasma CVD method, a thermal CVD method, an MOCVD method, and a photo CVD method. In contrast, the wet method include a spin coating method, an inkjet method a spray coating method, a stamp method, a microcontact printing method, a flexogaphic printing method, an offset printing method, a gravure printing method, a dipping method, and the like. For patterning, it is possible to use chemical etching such as shadow mask, laser transfer, and photolithography, and physical etching by ultraviolet light, laser, or the like, and the like. As a planarization technology, it is possible to use a laser planarization method, a fellow method, and the like.

Lastly, the on-chip lens layer 19 including a plurality of on-chip lenses 19L are disposed on the surface. Thus, the photoelectric conversion element 10 illustrated in FIG. 1 is completed.

In the photoelectric conversion element 10, in a case Where light enters the organic photoelectric converter 11G via the on-chip lenses 19L, the light passes through the organic photoelectric converter 11G and the inorganic photoelectric converters 11B and 11R in order, and each of green light, blue light, and red light is photoelectrically converted in the course of passing. In the following, signal acquisition operations of the respective colors are described.

### (Acquisition of Green Signal by Organic Photoelectric Converter 11G)

Of light having entered the photoelectric conversion element 10, first, green light is selectively detected (absorbed) and photoelectrically converted in the organic photoelectric converter 11G.

The organic photoelectric converter 11G is coupled to the gate Gamp of the amplifier transistor AMP and the floating diffusion FD3 via the through electrode 63. Thus, electrons of electron-hole pairs generated in the organic photoelectric converter 11G are extracted from the lower electrode 15 side, transferred to the second surface 11S2 side of the semiconductor substrate 11 via the through electrode 63, and accumulated in the floating diffusion FD3. Simultaneously with this, the amount of charges generated in the organic photoelectric converter 11G is modulated into voltage by the amplifier transistor AMP.

In addition, the reset gate Grst of the reset transistor RST is disposed adjacent to the floating diffusion FD3. Accordingly, the charges accumulated in the floating diffusion FD3 are reset by the reset transistor RST.

Herein, the organic photoelectric converter 11G is coupled not only to the amplifier transistor AMP but also to the floating diffusion FD3 via the through electrode 63, thus making it possible fur the reset transistor RST to easily reset the charges accumulated in the floating diffusion FD3.

In contrast to this, in a case where the through electrode 63 is not coupled to the floating diffusion FD3, it is difficult to reset the charges accumulated in the floating diffusion FD3, causing the charges to be drawn to the upper electrode 17 side by application of a large voltage. This nay damage the organic photoelectric conversion layer 16. In addition, a configuration that enables resetting in a short period of time causes an increase in dark time noise, thereby resulting in a trade-off; therefore, this configuration is difficult.

### (Acquisition of Blue Signal and Red Signal by Inorganic Photoelectric Converters 11B and 11R)

Subsequently, blue light and red light of the light having passed through the organic photoelectric converter 11G are absorbed and photoelectrically converted in sequence respectively in the inorganic photoelectric converter 11B and the inorganic photoelectric converter 11R. In the inorganic photoelectric converter 11B, electrons corresponding to the incident blue light are accumulated in the n region of the inorganic photoelectric converter 11B, and the accumulated electrons are transferred to the floating diffusion FD1 by the vertical transistor Tr1. Similarly, in the inorganic photoelectric converter 11R, electrons corresponding to the incident red light are accumulated in the n region of the inorganic photoelectric, converter 11R, and the accumulated electrons are transferred to the floating diffusion FD2 by the transfer transistor Tr2.

### 1-3. Workings and Effects

As described above, in recent years, various devices using organic thin films have been developed. The organic photoelectric conversion element is one of the devices, and an organic thin-film solar cell and an imaging element each using the organic photoelectric conversion element have been proposed. In particular, applications of the imaging element, not only to digital cameras and video camcorders but also to smartphone cameras, surveillance cameras, vehicle rear monitors, and collision prevention sensors, have widened and are receiving much attention. Accordingly, in order to be able to cope with any application, in the organic photoelectric conversion element included in the imaging element, an improvement in. performance is desired. Specifically, in addition to photoelectric conversion. efficiency, superior dark current characteristics and superior afterimage characteristics are desired.

In contrast, in the present embodiment, the organic photoelectric conversion layer 16 is formed using at least one kind of organic semiconductor material represented by the above-described general expression (1). Examples of the organic semiconductor material represented by the general expression (1) include a benzobisbenzothiophene (BBBT) derivative.

A mother skeleton of the BBBT derivative has ten positions into which a substituent group is allowed to be introduced. It was found from examples to be described later that introducing a substituent group into a 3-position and a 9-position (positions modified by A1 and A2 in the general expression (1)) of these positions made it possible to achieve superior dark current characteristics and superior afterimage characteristics in addition to favorable photoelectric conversion efficiency. The BBBT derivative in which the substituent groups are introduced into the 3-position and the 9-position has a linear molecular structure. Accordingly, in the organic photoelectric conversion layer 16, interference with intermolecular interaction between the BBBT derivatives by the substituent groups is reduced, and an orientation property of the BBBT derivative in the organic photoelectric conversion layer 16 is improved. As a result, carrier transportability in grains formed by the BBBT derivative is improved.

Moreover, in general, in the organic semiconductor material, the intermolecular interaction is moderately relaxed by adjusting a ratio of different kinds of elements in the mother skeleton. Actually, a grain size thrilled by the BBBT derivative becomes a moderate size, thereby forming a favorable (dense) film. For example, in a case where the organic photoelectric conversion layer 16 is formed using a subphthalocyanine derivative (light absorber) and fullerene C60 (n-type semiconductor), the grain size (particle diameter) formed by the p-type semiconductor. preferably smaller than 13 nm, and more preferably about 7 nm. In contrast, the BBBT derivative has a particle diameter of about 7 nm in an experimental example 3 to be described later. That is, the BBBT derivative has a favorable contact property (carrier transportability) between the grains thereof. Accordingly, for example, the organic photoelectric conversion layer 16 using the BBBT derivative makes it possible to improve carrier mobility between the grains irrespective of the presence or absence of any other organic semiconductor material.

Further, the mother skeleton of the BBBT derivative has an appropriate energy level to achieve favorable photoelectric conversion characteristics even in a case where the BBBT derivative is used in the organic photoelectric conversion layer 16 and a layer (for example, the buffer layers 16A and 16B) other than the organic photoelectric conversion layer 16. HOMO levels of the light absorber and an electron transporting material (n-type semiconductor) used in the organic photoelectric conversion layer are generally deeper than −6.2 eV. Accordingly, a hole transporting material used in the organic photoelectric conversion layer and an organic semiconductor material used in a buffer layer provided on the anode side preferably have a HOMO level shallow than −6.2 eV. This makes it possible to achieve favorable photoelectric conversion characteristics, favorable dark current characteristics, and favorable afterimage characteristics. Note that, in a case where the HOMO levels of the hole transporting material and the material of the buffer layer provided on the anode side are too shallow, a carrier path that becomes a dark current source is formed between the LL MO levels of the light absorber and the electron transporting material. Accordingly, the HOMO level of the hole transporting material is preferably, for example, deeper than −5.6 eV and shallower than −6.2 eV. It is to be noted that −5.6 eV is a value calculated on the basis of subphthalocyanine and a derivative thereof, and fullerene C60 and a derivative thereof. Meanwhile, the BBBT derivative represented by the above-described general expression (1) satisfies the above-described condition.

Furthermore, the mother skeleton of the BBBT derivative includes benzene and thiophene that are alternately condensed. An absorption wavelength of the mother skeleton is a short wavelength, and, for example, light absorptance in a visible region on a longer wavelength side than 450 nm is low. Accordingly, as with the imaging element including the photoelectric conversion element according to the present embodiment, in a longitudinal spectral type imaging element in which the organic photoelectric converter 11G and the inorganic photoelectric converters 11R and 11B are stacked, a degradation in photoelectric conversion efficiency of the inorganic photoelectric converters 11R and 11B disposed in lower layers with respect to a light incident direction is reduced.

As described above, the photoelectric conversion element 10 according to the present embodiment is formed using at least one kind of organic semiconductor material represented by the above-described general expression (1) such as a benzobisbenzothiophene (BBBT) derivative, which makes it possible to satisfy both favorable carrier transportability in the grains formed by the BBBT derivative and between the grains, and an appropriate energy level. This makes it possible to achieve favorable photoelectric conversion efficiency, superior dark current characteristics, and superior afterimage characteristics.

Further, in the present embodiment, as the material of the organic photoelectric conversion layer 16, subphthalocyanine or a derivative thereof and fullerene or a derivative thereof are used together with the BBBT derivative. This makes it possible to further improve the photoelectric conversion efficiency, the dark current characteristics, and the afterimage characteristics.

Next, description is given of modification examples (modification examples 1 and 2) of the present disclosure. It is to be noted that components corresponding to those of the photoelectric conversion element 10 according to the above-described embodiment are denoted by the same reference numerals, and description thereof is omitted.

### 2. MODIFICATION EXAMPLES

### 2-1. Modification Example 1

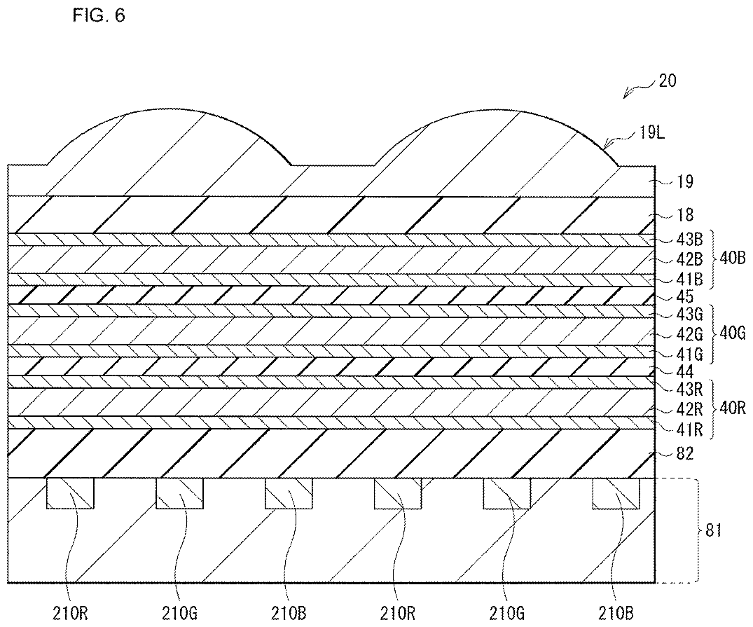

FIG. 6 illustrates a cross-sectional configuration of a photoelectric conversion element (Photoelectric conversion element 20) according to a modification example (modification example 1) of the present disclosure. The photoelectric conversion element 20 is an imaging element included in one unit pixel P of an imaging apparatus (imaging apparatus 1) such as a back-side illumination type CCD image sensor or a CMOS image sensor, as with the photoelectric conversion element 10 according to the above-described embodiment and the like. The photoelectric conversion element 20 according to the present modification example is a so-called longitudinal spectral system imaging element in which a red photoelectric converter 40R, a green photoelectric converter 40G, and a blue photoelectric converter 40B are stacked in this order on a silicon substrate 81 with an insulating layer 82 interposed therebetween.

The red photoelectric converter 40R, the green photoelectric converter 40G, and the blue photoelectric converter 40B respectively include organic photoelectric conversion layers 42R, 42G, and 42B between a pair of electrodes, specifically, between a first electrode 41R and a second electrode 43R, between a first electrode 41G and a second electrode 43G, and between a first electrode 41B and a second electrode 43B. In the present modification example, each of the organic photoelectric conversion layers 42R, 42G, and 42B has a configuration formed including the organic semiconductor material represented by the above-described general expression (1).

The photoelectric conversion element 20 has a configuration in which the red photoelectric converter 40R, the green photoelectric converter 40G, and the blue photoelectric converter 40B are stacked on the silicon substrate 81 with the insulating layer 82 interposed therebetween. The on-chip lenses 19L are provided on the blue photoelectric: converter 40B with the protective layer 18 and the on-chip lens layer 19 interposed therebetween. A red storage layer 210R, a green storage layer 210G, and a blue storage layer 210B are provided in the silicon substrate 81. Light having entered the on-chip lenses 19L is photoelectrically converted by the red photoelectric converter 40R, the green photoelectric converter 40G, and the blue photoelectric converter 40B, and signal charges are transmitted each from the red photoelectric converter 40R to the red storage layer 210R, from the green photoelectric converter 40G to the green storage layer 210G, and from the blue photoelectric converter 40B to the blue storage layer 210B. The signal charges may be electrons or holes generated by photoelectric conversion, but a case where electrons are read as signal charges is described as an example below.

The silicon substrate 81 includes, for example, a p-type silicon substrate. The red storage layer 210R, the green storage layer 210G, and the blue storage layer 210B provided in the silicon substrate 81 each include an n-type semiconductor region, and signal charges (electrons) supplied from the red photoelectric converter 40R, the green photoelectric converter 40G, and the blue photoelectric converter 40B are accumulated in the n-type semiconductor regions. The n-type semiconductor regions of the red storage layer 210R, the green storage layer 210G, and the blue storage layer 210B are formed by doping the silicon substrate 81 with an n-type impurity such as phosphorus (P) or arsenic (As), for example. It is to be noted that the silicon substrate 81 may be provided on a supporting substrate (not illustrated) including glass or the like.

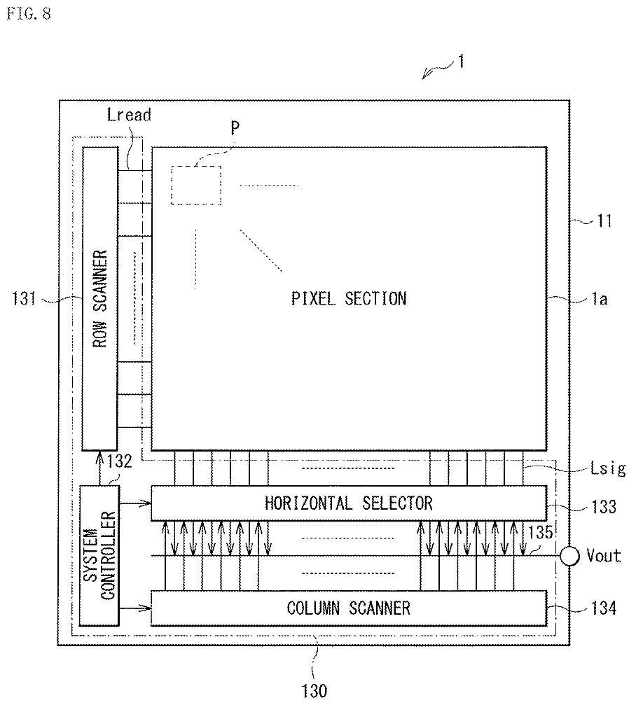

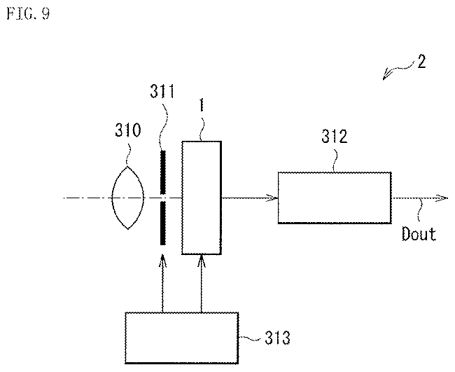

In the silicon substrate 81, a pixel transistor is provided. The pixel transfer is used to read electrons from each of the red storage layer 210R, the green storage layer 210G, and the blue storage layer 210B and transfer the electrons to a vertical signal line (vertical signal line Lsig in FIG. 9 to be described later), for example. A floating diffusion of the pixel transistor is provided in the substrate 81, and the floating diffusion is coupled to the red storage layer 210R, the green storage layer 210G, and the blue storage layer 210B. The floating diffusion includes an n-type semiconductor region.

The insulating layer 82 includes, for example, silicon oxide, silicon nitride, silicons oxynitride hafnium oxide, and the like. The insulating layer 82 may be configured by stacking a plurality of kinds of insulating films. The insulating layer 82 may include an organic insulating material. The insulating layer 82 includes respective plugs for coupling between the red storage layer 210R and the red photoelectric converter 40R, between the green storage layer 210G and the green photoelectric converter 40G, and between the blue storage layer 210B and the blue photoelectric converter 40B, and electrodes.

The red photoelectric converter 40R includes the first electrode 41R, the organic photoelectric conversion layer 42R, and the second electrode 43R in this order from a position close to the silicon substrate 81. The green photoelectric converter 40G includes the first electrode 41G, the organic photoelectric conversion layer 42G, and the second electrode 43G in this order from a position close to the red photoelectric converter 40R. The blue photoelectric converter 40B includes the first electrode 41B, the organic photoelectric conversion layer 42B, and the second electrode 43B in this order from a position close to the green photoelectric converter 40G. An insulating layer 44 is provided between the red photoelectric converter 40R and the green photoelectric converter 40G, and an insulating layer 45 is provided between the green photoelectric converter 40G and the blue photoelectric converter 40B. The red photoelectric converter 40R, the green photoelectric converter 40G, and the blue photoelectric converter 40B respectively selectively absorb red (for example, a wavelength of 620 nm or greater and less than 750 nm) light, green (for example, a wavelength of 450 nm or greater and less than 650 nm, more preferably 495 nm or greater and less than 620 nm) light, and blue (for example, a wavelength of 425 or greater and less than 495 nm) light to generate electron-hole pairs.

The first electrode 41R, the first electrode 41G, and the first electrode 41B respectively extract signal charges generated in the organic photoelectric conversion layer 42R, signal charges generated in the organic photoelectric conversion layer 42G, and signal charges generated in the organic photoelectric conversion layer 42B. The first electrodes 41R, 41G, and 41B are provided for each pixel, for example. The first electrodes 41R, 41G, and 41B each include, for example, an electrically conductive film having light transmissivity similarly to the lower electrode 15 in the above-described embodiment. A thickness of each of the first electrodes 41R, 41G, and 41B is, for example, from 20 nm to 200 nm both inclusive, and preferably from 30 nm to 100 nm both inclusive.

A buffer layer may be provided each between the first electrode 41R and the organic photoelectric conversion layer 42R, between the first electrode 41G and the organic photoelectric conversion layer 42G, and between the first electrode 41B and the organic photoelectric conversion layer 42B, for example. The buffer layer serves to promote supplying of carriers generated in the organic photoelectric conversion layers 42R, 42G, and 42B to the first electrodes 41R, 41G, and 41B, and in a case where the photoelectric conversion element 20 is of an electron readout system, it is possible to use a material used in the buffer layer 16A in the above-described embodiment. In addition, in a case of a hole readout system, it is possible to use a material used in the buffer layer 16B in the above-described embodiment.

The organic photoelectric conversion layers 42R, 42G, and 42B each absorb light in the above-described selective wavelength region for photoelectric conversion, and allow light in another wavelength region to pass therethrough. A thickness of each of the organic photoelectric conversion layers 42R, 42G, and 42B is, for example, from 100 nm to 300 nm both inclusive.

As with the organic photoelectric conversion layer 16 in the above-described embodiment, the organic photoelectric conversion layers 42R, 42G, and 42B each include, for example, two or more types of organic semiconductor materials, and preferably includes, for example, one or both of a p-type semiconductor and an n-type semiconductor. For example, in case there each of the organic photoelectric conversion layers 42R, 42G, and 42B includes two kinds of organic semiconductor materials, that is, the p-type semiconductor and the n-type semiconductor; for example, one of the p-type semiconductor and the n-type semiconductor is preferably a material having transmissivity to visible light, and the other is preferably a material that performs photoelectric conversion of light in a selective wavelength region (for example, from 450 nm to 650 nm both inclusive). Alternatively, each of the organic photoelectric conversion layers 42R, 42G, and 42B preferably includes three kinds of organic semiconductor materials, that is, a material (light absorber) that performs photoelectric. conversion of light in a selective wavelength region, and the n-type semiconductor and the p-type semiconductor having transmissivity to visible light. In the present modification example, each of the organic photoelectric conversion layers 42R, 42G, and 42B includes, as the p-type semiconductor, one or more kinds of organic. semiconductor materials (for example, a BBBT derivative) represented by the above-described general expression (1).

The organic photoelectric: conversion layers 42R, 42G, and 42B preferably use fullerene C60 represented by the above-described general expression (2) or a derivative thereof, or fullerene C70 represented by the above-described general expression (3) or a derivative thereof, in addition to the BBBT derivative. Using at least one kind of fullerene C60, fullerene C70, or a derivative thereof makes it possible to further improve photoelectric conversion efficiency and reduce a dark current.

The organic photoelectric conversion layers 42R, 42G, and 42B preferably further use a material (light absorber) that is allowed to perform photoelectric conversion of light in the above-described selective wavelength region. This makes it possible to selectively perform photoelectric conversion of red light, green light, and blue light respectively by the organic photoelectric conversion layer 42R, the organic photoelectric conversion layer 42G and the organic photoelectric conversion layer 42B. Examples of such a material in the organic photoelectric conversion layer 42R include subnaphthalocyanine or a derivative thereof, and phthalocyanine or a derivative thereof. Examples of such a material in the organic photoelectric conversion layer 42G include subphthalocyanine or a derivative thereof, and the like. Examples of such a material in the organic photoelectric conversion layer 42B include coumarin or a derivative, and porphyrin or a derivative thereof.

It is to be noted that the BBBT derivative, subphthalocyanine or a derivative thereof, naphthalocyanine or a derivative thereof, and fullerene or a derivative thereof function as a p-type semiconductor or an n-type semiconductor depending on materials to be combined together.

For example, a buffer layer may be provided each between the organic photoelectric conversion layer 42R and the second electrode 43R, between the organic photoelectric conversion layer 42G and the second electrode 43G, and between the organic photoelectric conversion layer 42B and the second electrode 43B, similarly between the first electrode 41R and the organic photoelectric conversion layer 42R, and the like. As a constituent material of the buffer layer, it is possible to use a material used in the buffer layer 16A in the above-described embodiment in a case where the photoelectric conversion element 20 is of the electron readout system. In addition, in a case of the hole readout system, it is possible to use a material used in the buffer layer 16B in the above-described embodiment.

The second electrode 43R, the second electrode 43G, the second electrode 43B respectively serve to extract holes generated in the organic photoelectric conversion layer 42R, holes generated in the organic photoelectric conversion layer 42G, and holes generated in the organic photoelectric conversion layer 42B. The holes extracted from the second electrodes 43R, 43G, and 43B are discharged to, for example, the p-type semiconductor region (not illustrated) in the silicon substrate 81 through various transmission paths (not illustrated). The second electrodes 43R, 43G, and 43B include, for example, an electrically conductive material such as gold, silver, copper, and aluminum. As with the first electrodes 41R, 41G, and 41B for example, the second electrodes 43R, 43G, and 43B may include, for example, an electrically conductive film having light transmissivity similarly to the low electrode 15 in the above-described embodiment. The holes extracted from the second electrodes 43R 43G, and 43B are discharged; therefore, in a case where a plurality of photoelectric :conversion elements 20 is disposed in the imaging apparatus 1 to be described later, the second electrodes 43R, 43G, and 43B may be provided common to each of the photoelectric conversion elements 20 (unit pixels P). A thickness of each of the second electrodes 43R, 43G, and 43B is, for example, form 20 nm to 200 nm both inclusive, and preferably from 30 nm to 100 nm both inclusive.

The insulating layer 44 serves to insulate the second electrode 43R and the first electrode 41G from each other, and the insulating layer 45 serves to insulate the second electrode 43G and the first electrode 41B from each other. The insulating. layers 44 and 45 include, for example, a metal oxide, a metal sulfide, or an organic substance. Examples of the metal oxide include silicon oxide, aluminum oxide, zirconium oxide, titanium oxide, zinc oxide, tungsten oxide, magnesium oxide, niobium oxide, tin oxide, gallium oxide, and the like. Examples of the metal sulfide include zinc sulfide, magnesium sulfide, and the like. A band gap of a constituent material of each of the insulating, layers 44 and 45 is preferably 3.0 eV or greater. A thickness of each of the insulating layers 44 and 45 is, for example, from 2 nm to 100 nm both inclusive.

As described above, in the present modification example, the organic photoelectric conversion layers 42R (and 42G and 42B) are each configured using the organic semiconductor material represented by the above-described general expression. (1) such as the BBBT derivative, for example. Accordingly, as with the above-described embodiment, interference with intermolecular interaction in the organic semiconductor material represented by the above-described general expression (1) is reduced, and an orientation property of the organic semiconductor material represented by the above-described general expression (1) in the organic photoelectric conversion layers 42R (and 42G, and 42B) is improved. In addition, as with the above-described embodiment, favorable carrier transportability and an appropriate energy level are compatible in grains formed by the organic semiconductor material represented by the general expression (1) and between the grains, which makes it possible to achieve favorable photoelectric conversion efficiency, superior dark current characteristics, and superior afterimage characteristics.

It is to be noted that, in the present modification example, an example in which the organic semiconductor material represented by the general expression (1) such as the BBBT derivative is used in the organic photoelectric conversion layers 42R (and 42G and 42B) is described but this is not limitative. Even using the organic semiconductor material in an organic layer provided between the first electrodes 41R (and 41G and 41B) and the second electrode 43R (and 43G and 4B) in addition to the organic photoelectric conversion layers 42R (and 42G and 42B) makes it possible to achieve effects similar to those in the present modification example.

### 2-7. Modification Example 2

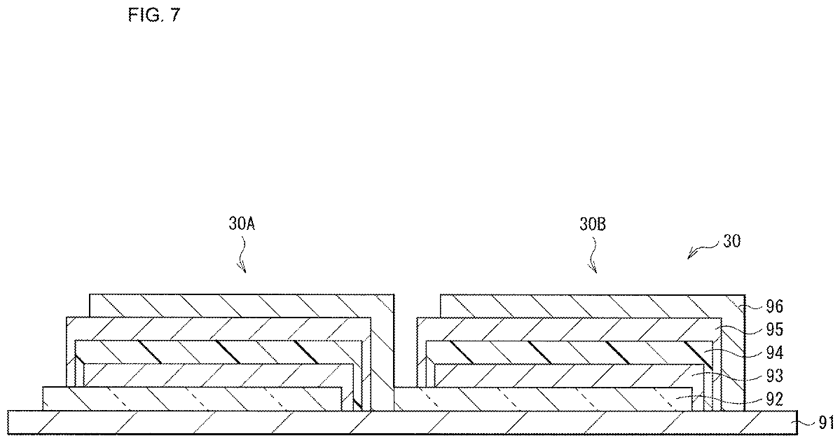

FIG. 7 illustrates an example of a cross-sectional configuration of an organic solar cell module (solar cell 30) including photoelectric conversion elements 30A and 30B according to a modification example (modification example 2) of the present disclosure. The photoelectric conversion elements 30A and 30B according to the present modification example each have a configuration in which a transparent electrode 92, a hole transport layer 93, an organic photoelectric conversion layer 94, an electron transport layer 95, and a counter electrode 96 are stacked on a substrate 91. The photoelectric conversion elements 30A and 30B according to the present modification example have a configuration in which the organic photoelectric conversion layer 94 is formed including the organic semiconductor material represented by the above-described general expression (1) (for example, a BBBT derivative).

The substrate 91 serves to retain respective layers (for example, the organic photoelectric conversion layer 94) included in the photoelectric conversion elements 30A and 30B, and includes, for example, a plate-like member having two main surfaces opposed to each other. As the substrate 91, organic polymers such as polymethyl methacrylate (PMMA), polyvinyl alcohol (PVA), polyvinyl phenol (PVP), polyethersulfone (PES), polyimide, polycarbonate (PC), polyethylene terephthalate (PET), and polyethylene naphthalate (PEN) are used. These organic polymers form flexible substrates such as a plastic film, a plastic sheet, and a plastic substrate. Using these flexible substrates allows for incorporation or integration into an electronic substrate having a curved shape, for example. In addition to these substrates, various kinds of glass substrates, various kinds of glass substrates having a surface on which an insulating film is formed, a quartz substrate, a quartz substrate having a surface on which an insulating film is formed, a silicon semiconductor substrate, and a metal substrate that has a surface on which an insulating film is formed and includes various kinds of alloys such as stainless steel or various kinds of metals are used. It is to be noted that the insulating film formed on any of the above-described substrates include a silicon oxide-based material (for example, SiO_{X} or spin-on glass (SOG)), silicon nitride (SiNₓ), silicon oxynitride (SiON), a metal oxide such as aluminum oxide (Al₂O₃), or a metal salt. In addition, an insulating organic substance film may be formed. Examples of an insulating organic substance material include a polyphenol-based material, a polyvinyl phenol-based material, a polyimide-based material, a polyamide-based material, a polyamide imide-based material, a fluorine-based polymer material, a borazine-silicon polymer material, a truxene-based material, and the like that are allowed to be subjected to lithography. Further, it is also possible to use an electrically conductive substrate having a surface on which these insulating films are formed, for example, a substrate including a metal such as gold and aluminum, a substrate including highly oriented graphite, and the like.

It is to be noted that the surface of the substrate 91 is desirably smooth, but the surface may have surface roughness to such a degree as not to adversely affect characteristics of the organic photoelectric conversion layer 94. Further, on the surface of the substrate, a silanol derivative by a silane coupling method may be formed, a thin film including a thiol derivative, a carboxylic acid derivative, a phosphoric acid derivative, and the like by a SAM method and the like may be formed, or a thin film including an insulating metal salt or an insulating metal complex by a CVD method and the like may be formed. This causes an improvement in adhesion between the substrate 91 and the transparent electrode 92.

The transparent electrode 92 includes, for example, an electrically conductive film having light transmissivity similarly to the lower electrode 15 in the above-described embodiment. A thickness of each of the first electrodes 41R, 41G, and 41B is, for example, from 20 nm to 200 nm both inclusive, and preferably from 30 nm to 100 nm both inclusive.

The hole transport layer 93 serves to efficiently extract charges (herein, holes) generated in the organic photoelectric conversion layer 94. Examples of a material included in the hole transport layer 93 include PEDOT such as BaytronP (registered trademark) manufactured by H. C. Starck-V TECH Ltd., polyaniline and a doping material thereof, a cyan compound described in WO2006/019270, and the like. As a method of forming the hole transport layer 93, any method of a vacuum evaporation method and a coating method may be used, but the coating method is preferable. A reason for this is that a coating film is formed below the organic photoelectric conversion layer 9 before forming the organic photoelectric conversion layer 94, which causes an effect of leveling a coating surface, thereby making it possible to reduce an influence of leakage and the like. It is to be noted that as a material of the hole transport layer 93, the material of the buffer layer 16B described in the above-described embodiment may be used.

The organic photoelectric conversion layer 94 includes, for example, two or more kinds of organic semiconductor materials, as with the organic photoelectric conversion layers 16, 42R, 42G, and 42B in the above-described embodiment and the modification example 1, and preferably includes, for example, one or both of the p-type semiconductor and the n-type semiconductor, For example, in a case Where the organic photoelectric conversion layer 94 includes two kinds of organic semiconductor materials, that is, the p-type semiconductor and the n-type semiconductor, and one of the p-type semiconductor and the n-type semiconductor is preferably a material having transmissivity to visible light, and the other is preferably a material that performs photoelectric conversion of light in a visible region and a near-infrared region (for example, from 400 nm to 1300 nm both inclusive) Alternatively, the organic photoelectric conversion layer 94 preferably includes three kinds of organic semiconductor materials, that is, a material (light absorber) that performs photoelectric conversion of light in a. visible region and a near-infrared region, and the n-type semiconductor and the p-type semiconductor having trasmissivity to visible light. In the present modification example, the organic photoelectric conversion layer 94 includes, as the p-type semiconductor, one or more kinds of organic semiconductor materials (for example, a BBBT derivative) represented by the above-described general expression (1).

The organic photoelectric conversion layer 94 preferably uses fullerene C60 represented by the above-described general expression (2) or a derivative thereof or fullerene C70 represented by the above-described general expression (3) or a derivative thereof, in addition to the BBBT derivative, Using at least one kind of fullerene C60, fullerene C70, or a derivative thereof makes it possible to further improve photoelectric conversion efficiency. Further, the organic photoelectric conversion layer 94 preferably uses the material (light absorber) that performs photoelectric conversion of light in the visible region and the near-infrared region, and examples of such a material include subphthalocyanine represented by the above-described general expression (4) or a derivative thereof.

The electron transport layer 95 serves to efficiently extract charges (herein, electrons) generated in the organic photoelectric conversion layer 94. Examples of a material included in the electron transport layer 95 include octaazaporphyrin and a perfluoro form of a p-type semiconductor material (such as perfluoropentacene and perfluorophthalocyanine). As a method of forming the electron transport layer 95, any method of a vacuum evaporation and a coating method may be used, but the coating method is preferable.

The counter electrode 96 includes, for example, an electrically conductive film having light transmissivity similarly to the lower electrode 15 in the above-described embodiment. A thickness of each of the first electrodes 41R, 41G, and 41B is, for example, from 20 nm to 200 nm both inclusive, and preferably from 30 nm to 100 nm both inclusive.

It is to be noted that the buffer layers 16A and 16B described in the above-described embodiment may be respectively provided between the organic photoelectric conversion layer 94 and the transparent electrode 92 and between the organic photoelectric conversion layer 94 and the counter electrode 96, in addition to the hole transport layer 93 and the electron transport layer 95.

The solar cell 30 in the present modification example includes two photoelectric conversion elements 30A and 30B arranged in a lateral direction, and the counter electrode 96 of the photoelectric conversion element 30A on the left in the drawing and the transparent electrode 92 of the photoelectric conversion element 30B on the right are coupled to each other in series, which makes it possible to construct an organic solar cell module having a serial structure and having high electromotive force. In the present modification example, two photoelectric conversion elements 30A and 30B are coupled to each other in series; however, the number of elements coupled to each other in series is not limited to two, and it is possible to provide additional elements as appropriate in accordance with specifications of an organic module. It is to be noted that sealing by a gas-barrier film may be performed on the surfaces of the photoelectric conversion elements 30A and 30B.

As described above, the organic photoelectric conversion layer 94 is configured using the organic semiconductor material represented by the above-described general expression (1) such as the BBBT derivative. This makes it possible to reduce interference with intermolecular interaction in the organic semiconductor material represented by the above-described general expression (1), and to improve an orientation property in the organic photoelectic conversion layer 94. In addition, as with the above-described embodiment, favorable carrier transportability and an appropriate energy level are compatible in grains formed by the organic semiconductor material represented by the general expression (1) and between the grains, which makes it possible to provide the solar cell 30 having favorable photoelectric conversion efficiency, superior dark current characteristics, and superior afterimage characteristics.

It is to be noted that, in the present modification example, an example in which the organic semiconductor material represented by the above-described general expression (1) such as the BBBT derivative is used in the organic photoelectric conversion layer 94 is described, but this is not limitative. Even using the organic semiconductor material in an organic layer provided between the transparent electrode 92 and the counter electrode 96, for example, the hole transport layer 93 and the electron transport layer 95 in addition to the organic photoelectric conversion layer 94 makes it possible to achieve effects similar to those in the present modification example.

### 3. APPLICATION EXAMPLE

### Application Example 1

FIG. 8 illustrates an overall configuration of the imaging apparatus 1 using, for each of the pixels, the photoelectric conversion element 10 described in the above-described embodiment. The imaging apparatus 1 is a CMOS image sensor, and includes, on the semiconductor substrate 11, a pixel section la as an imaging region and a peripheral circuit section 130 including, for example, a row scanner 131, a horizontal selector 133, a column scanner 134, and a system controller 132 in a peripheral region of the pixel section 1a.

The pixel section 1a has a plurality of unit pixels P (each corresponding to the photoelectric conversion element 10) two-dimensionally arranged in a matrix, for example. The unit pixels P are wired with pixel drive lines Lread (specifically, row selection lines and reset control lines) for respective pixel rows, and vertical signal lines Lsig for respective pixel columns, for example. The pixel drive lines Lread transmit drive signals for signal reading from the pixels. The pixel drive lines Lread each have one end coupled to a corresponding one of output terminals, corresponding to the respective rows, of the row scanner 131.

The row seamier 131 includes a shift register, an address decoder; and the like, and is a pixel driver, for example, that drives the respective unit pixels P the pixel section la on a row-by-row basis. A signal outputted from each of the unit pixels P of a pixel row selectively scanned by the row scanner 131 is supplied to the horizontal selector 133 through each of the vertical signal lines Lsig. The horizontal selector 133 includes an amplifier, a horizontal selection switch, and the like provided for each of the vertical signal lines Lsig.

The column scanner 134 includes a shift register, an address decoder, and the like, and drives respective horizontal selection switches of the horizontal selector 133 in sequence while scanning the horizontal selection switches. Such selective scanning by the column scanner 134 causes the signals of the respective pixels transmitted through the respective vertical signal lines Lsig to be outputted in sequence to a horizontal signal line 135 and thereafter transmitted to outside of the semiconductor substrate 11 through the horizontal signal line 135.

Circuit components including the row scanner 131, the horizontal selector 133, the column scanner 134, and the horizontal signal line 135 may be formed directly on the semiconductor substrate 11 or disposed in an external control IC. Alternatively, these circuit components may be formed in any other substrate coupled by a cable, or the like.

The system controller 132 receives a clock given from the outside of the semiconductor substrate 11, or data or the like on instructions of operation modes, and also outputs data such as internal information of the imaging apparatus 1. The system controller 132 further has a timing generator that generates various timing signals, and performs drive control of the peripheral circuits such as the row seamier 131, the horizontal selector 133. and the column scanner 134, on the basis of the various timing signals generated by the timing generator.

### Application Example 2

The above-described imaging apparatus 1 is applicable to, for example, various kinds of electronic apparatuses (imaging apparatuses) having imaging functions. Examples of the electronic apparatuses include camera systems such as digital still cameras and video cameras and mobile phones having the imaging functions. FIG. 9 illustrates, for purpose of an example, a schematic configuration of a camera 2. The camera 2 is a video camera that enables shooting of a still image or a moving image, fur example, and includes the imaging apparatus 1, an optical system (optical lens) 310, a shutter apparatus 311, a driver 313 that drives the imaging apparatus 1 and the shutter apparatus 311, and a signal processor 312.

The optical system 310 guides image light (incident light) from an object the pixel section 1a of the imaging apparatus 1. The optical system 310 may include a plurality of optical lenses. The shutter apparatus 311 controls a period in which the imaging apparatus 1 is irradiated with the light and a period in which the light is blocked. The driver 313 controls a transfer operation of the imaging apparatus 1 and a shutter operation of the shutter apparatus 311. The signal processor 312 performs various types of signal processing on signals outputted from the imaging apparatus 1. An image signal Lout having been subjected to the signal processing is stored in a storage medium such as a memory or outputted to a monitor, or the like.

### Application Example 3

### <Example of Application to In-Vivo Information Acquisition System

The technology (present technology) according to the present disclosure is applicable to various products. For example, the technology according to the present disclosure may be applied to an endoscopic surgery system.

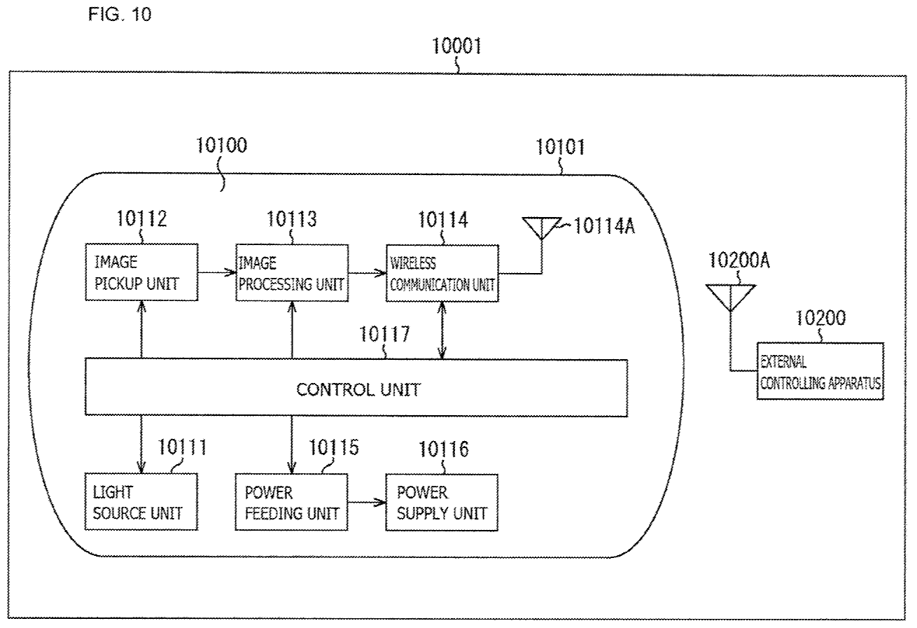

FIG. 10 is a block diagram depicting an example of a schematic configuration of an in-vivo information acquisition system of a patient using a capsule type endoscope, to which the technology according to an embodiment of the present disclosure (present technology) can be applied.

The in-vivo information acquisition system 10001 includes a capsule type endoscope 10100 and an external controlling apparatus 10200.

The capsule type endoscope 10100 is swallowed by a patient at the time of inspection. The capsule type endoscope 10100 has an image pickup function and a wireless communication function and successively picks up an image of the inside of an organ such as the stomach or an intestine (hereinafter referred to as in-vivo image) at predetermined intervals while it moves inside of the organ by peristaltic motion for a period of time until it is naturally discharged from the patient. Then, the capsule type endoscope 10100 successively transmits information of the in-vivo image to the external controlling apparatus 10200 outside the body by wireless transmission.

The external controlling apparatus 10200 integrally controls operation of the in-vivo information acquisition system 10001. Further, the external controlling apparatus 10200 receives information of an in-vivo image transmitted thereto from the capsule type endoscope 10100 and generates image data for displaying the in-vivo image on a display apparatus (not depicted) on the basis of the received information of the in-vivo image.

In the in-vivo information acquisition system 10001, an in-vivo image imaged a state of the inside of the body of a patient can be acquired at any time in this manner for a period of time until the capsule type endoscope 10100 is discharged after it is swallowed.

A configuration and functions of the capsule type endoscope 10100 and the external controlling apparatus 10200 are described in more detail below

The capsule type endoscope 10100 includes a housing 10101 of the capsule type, in which a light source unit 10111, an image pickup unit 10112, an image processing unit 10113, a wireless communication unit 10114, a power feeding unit 10115, a power supply unit 10116 and a control unit 10117 are accommodated.

The light source unit 10111 includes a light source such as, for example, a light emitting diode (LED) and irradiates light on an image pickup field-of-view of the image pickup unit 10112.

The image pickup unit 10112 includes an image pickup element and an optical system including a plurality of lenses provided at a preceding stage to the image pickup element. Reflected light (hereinafter referred to as observation light) of light irradiated on a body tissue which is an observation target is condensed by the optical system and introduced into the image pickup element. In the image pickup unit 10112, the incident observation light is photoelectrically converted by the image pickup element, by which an image signal corresponding to the observation light is generated. The image signal generated by the image pickup unit 10112 is provided to the image processing unit 10113.

The image processing unit 10113 includes a processor such as a central processing unit (CPU) or a graphics processing unit (GPU) and performs various signal processes for an image signal generated by the image pickup unit 10112. The image processing unit 10113 provides the image signal for which the signal processes have been performed thereby as RAW data to the wireless communication unit 10114.

The wireless communication unit 10114 performs a predetermined process such as a modulation process for the image signal for which the signal processes have been performed by the image processing unit 10113 and transmits the resulting image signal to the external controlling apparatus 10200 through an antenna 10114A. Further, the wireless communication unit 10114 receives a control signal relating to driving control of the capsule type endoscope 10100 from the external controlling apparatus 10200 through the antenna 10114A. The wireless communication unit 10114 provides the control signal received from the external controlling apparatus 10200 to the control unit 10117.

The power feeding unit 10115 includes an antenna coil for power reception, a power regeneration circuit f©r regenerating, electric power from current generated in the antenna coil, a voltage booster circuit and so forth. The power feeding unit 10115 generates electric power using the principle of non-contact charging.

The power supply unit 10116 includes a secondary battery and stores electric power generated by the power feeding unit 10115. In FIG. 10, in order to avoid complicated illustration, an arrow mark indicative of a supply destination of electric power from the power supply unit 10116 and so forth are omitted. However, electric power stored in the power supply unit 10116 is supplied to and can be used to drive the light source unit 10111, the image pickup unit 10112, the image processing unit 10113, the wireless communication unit 10114 and the control unit 10117.

The control unit 10117 includes a processor such as a CPU and suitably controls driving of the light source unit 10111, the image pickup unit 10112, the image processing unit 10113, the wireless communication unit 10114 and the power feeding unit 10115 in accordance with a control signal transmitted thereto from the external controlling apparatus 10200.

The external controlling apparatus 10200 includes a processor such as a CPU or a GPU, a microcomputer, a control board or the like in which a processor and a storage element such as a memory are mixedly incorporated. The external controlling apparatus 10200 transmits a control signal to the control unit 10117 of the capsule type endoscope 10100 through an antenna 10200A to control operation of the capsule type endoscope 10100. In the capsule type endoscope 10100, an irradiation condition of light upon an observation target of the light source unit 10111 can be changed, for example, in accordance with a control signal from the external controlling apparatus 10200. Further, an image pickup condition (for example, a frame rate, an exposure value or the like of the image pickup unit 10112) can be changed in accordance with a control signal from the external controlling apparatus 10200. Further, the substance of processing by the image processing unit 10113 or a condition for transmitting an image signal from the wireless communication unit 10114 (for example, a transmission interval, a transmission image number or the like) may be changed in accordance with a control signal from the external controlling apparatus 10200.

Further, the external controlling apparatus 10200 performs various image processes for an image signal transmitted thereto from the capsule type endoscope 10100 to generate image data for displaying a picked up in-vivo image on the display apparatus. As the image processes, various signal processes can be performed such as, for example, a development process (demosaic process), an image quality improving process (bandwidth enhancement process, a super-resolution process, a noise reduction (NR) process and/or image stabilization process) and/or an enlargement process (electronic zooming process). The external controlling apparatus 10200 controls driving of the display apparatus to cause the display apparatus to display a picked up in-vivo image on the basis of generated image data. Alternatively, the external controlling apparatus 10200 may also control a recording apparatus (not depicted) to record generated image data or control a printing apparatus (not depicted) to output generated image data by printing.

One example of the in-vivo information acquisition system to which the technology according to the present disclosure is applicable has been described above. The technology according to the present disclosure is applicable to, for example, the image pickup unit 10112 of the configurations described above. This makes it possible to improve accuracy of an inspection.

### Application Example 4

### <4. Example of Application to Endoscopic Surgery System>

The technology (present technology) according to the present disclosure is applicable to various products. For example, the technology according to the present disclosure may be applied to an endoscopic surgery system.

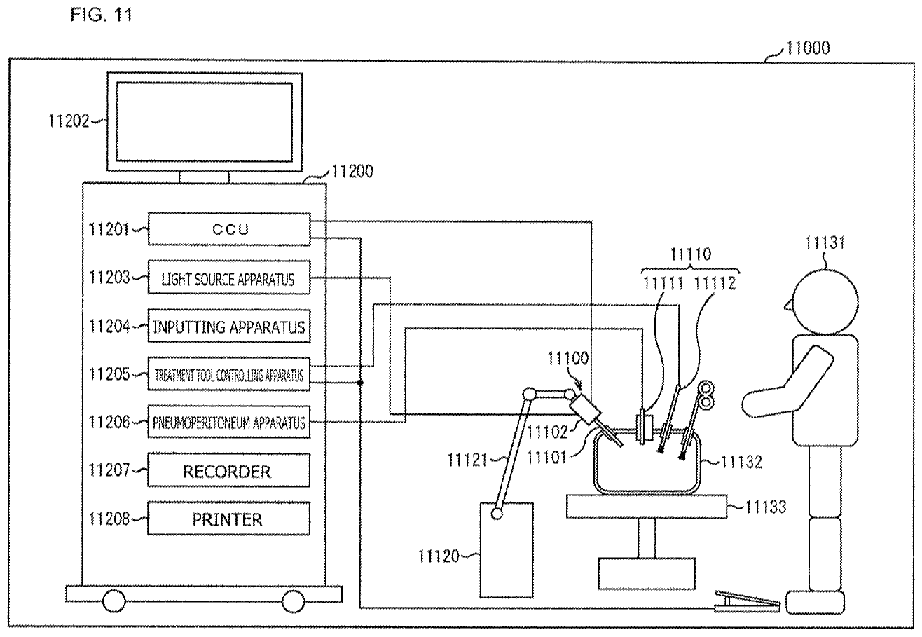

In FIG. 11 is a view depicting an example of a schematic configuration of an endoscopic surgery system to which the technology according to an embodiment of the present disclosure (present technology) can be applied.

In FIG. 11, a state is illustrated in which a surgeon (medical doctor) 11131 is using an endoscopic surgery system 11000 to perform surgery for a patient 11132 on a patient bed 11133. As depicted, the endoscopic surgery system 11000 includes an endoscope 11100, other surgical tools 11110 such as a pneumoperitoneum tube 11111 and an energy device 11112, a supporting arm apparatus 11120 which supports the endoscope 11100 thereon, and a cart 11200 on which various apparatus for endoscopic surgery are mounted.

The endoscope 11100 includes a lens barrel 11101 having a region of a predetermined length from a distal end thereof to be inserted into a body cavity of the patient 11132, and a camera head 11102 connected to a proximal end of the lens barrel 11101. In the example depicted, the endoscope 11100 is depicted which includes as a rigid endoscope having the lens barrel 11101 of the hard type. However, the endoscope 11100 may otherwise be included as a flexible endoscope having the lens barrel 11101 of the flexible type.

The lens barrel 11101 has, at a distal end thereof, an opening in which an objective lens is fitted. A light source apparatus 11203 is connected to the endoscope 11100 such that light generated by the light source apparatus 11203 is introduced to a distal end of the lens barrel 11101 by a light guide extending in the inside of the lens barrel 11101 and is irradiated toward an observation target in a body cavity of the patient 11132 through the objective lens. It is to be noted that the endoscope 11100 may be a forward-viewing endoscope or may be an oblique-viewing endoscope or a side-viewing endoscope.

An optical system and an image pickup element are provided in the inside of the camera head 11102 such that reflected light (observation light) from the observation target is condensed on the image pickup element by the optical system. The observation light is photo-electrically converted by the image pickup element to generate an electric signal corresponding to the observation light, namely, an image signal corresponding to an observation image. The image signal is transmitted as RAW data to a CCU 11201.

The CCU 11201 includes a central processing unit (CPU), a graphics processing unit (GPU) or the like and integrally controls operation of the endoscope 11100 and a display apparatus 11202. Further, the CCU 11201 receives an image signal from the camera head 11102 and performs, for the image signal, various image processes for displaying an image based on the image signal such as, for example, a development process (demosaic process).

The display apparatus 11202 displays thereon an image based on an image signal, for which the image processes have been performed by the CCU 11201, under the control of the CCU 11201.

The light source apparatus 11203 includes a light source such as, for example, a light emitting diode (LED) and supplies irradiation light upon imaging of a surgical region to the endoscope 11100.

An inputting apparatus 11204 is an input interface for the endoscopic surgery system 11000. A user can perform inputting of various kinds of information or instruction inputting to the endoscopic surgery system 11000 though the inputting apparatus 11204. For example, the user would input an instruction or a like to change an image pickup condition (type of irradiation light, magnification, focal distance or the like) by the endoscope 11100.

A treatment tool controlling apparatus 11205 controls driving of the energy device 11112 for cautery or incision of a tissue, sealing of a blood vessel or the like. A pneumoperitoneum apparatus 11206 feeds. gas into a body cavity of the patient 11132 through the pneumoperitoneum tube 11111 to inflate the body cavity in order to secure the field of view of the endoscope 11100 and secure the working space for the surgeon. A recorder 11207 is an apparatus capable of recording various kinds of information relating to surgery. A printer 11208 is an apparatus capable of printing various kinds of information relating to surgery in various forms such as a text, an image or a graph.

It is to be noted that the light source apparatus 11203 which supplies irradiation light when a surgical region is to be imaged to the endoscope 11100 may include a white light source which includes, for example, an IFD, a laser light source or a combination of them. Where a white light source includes a combination of red, green, and blue (ROB) laser light sources, since the output intensity and the output timing can be controlled with a high degree of accuracy for each color (each wavelength), adjustment of the white balance of a picked up image can be performed by the light source apparatus 11203. Further, in this case, if laser beams from the respective ROB laser light sources are irradiated time-divisionally on an observation target and driving of the image pickup elements of the camera, head 11102 are controlled in synchronism with the irradiation timings. Then images individually corresponding to the R, G and B colors can be also picked up time-divisionally. According to this method, a color image can be obtained even if color filters are not provided for the image pickup element.

Further, the light source apparatus 11203 may be controlled such that the intensity of light to be outputted is changed for each predetermined time. By containing driving of the image pickup element of the camera head 11102 in synchronism with the timing of the change of the intensity of light to acquire images time-divisionally and synthesizing the images, an image of a high dynamic range free from underexposed blocked up shadows and overexposed highlights can be created.

Further, the light source apparatus 11203 may be configured to supply light of a predetermined wavelength band ready for special light observation. In special light observation, for example, by utilizing the wavelength dependency of absorptance of light in a body tissue to irradiate light of a narrow band in comparison with irradiation light upon ordinary observation (namely, white light), narrow band observation (narrow band imaging) of imaging a predetermined tissue such as a blood vessel of a superficial portion of the mucous membrane or the like in a high contrast is performed. Alternatively, in special light observation, fluorescent observation for obtaining an image from fluorescent light generated by irradiation of excitation light may be performed. In fluorescent observation, it is possible to perform observation of fluorescent light from a body tissue by irradiating excitation light on the body tissue (autofluorescence observation) or to obtain a fluorescent light image by locally injecting a reagent such as indocyanine green (ICG) into a body tissue and irradiating excitation light corresponding to a fluorescent light wavelength of the reagent upon the body tissue. The light source apparatus 11203 can be configured to supply such narrow-band light and/or excitation light suitable for special light observation as described above.

FIG. 12 is a block diagram depicting an example of a functional configuration of the camera head 11102 and the CCU 11201 depicted in FIG. 11.

The camera head 11102 includes a lens unit 11401, an image pickup unit 11402, a driving unit 11403, a communication unit 11404 and a camera head controlling unit 11405, The CCU 11201 includes a communication unit 11411 an image processing unit 11412 and a control unit 11413. The camera head 11102 and the CCU 11201 are connected for communication to each other by a transmission cable 11400.

The lens unit 11401 is an optical system, provided at a connecting location to the lens barrel 11101. Observation light taken in from a distal end of the lens barrel 11101 is guided to the camera head 11102 and introduced into the lens unit 11401. The lens unit 11401 includes a combination of a plurality of lenses including a zoom lens and a focusing lens.

The number of image pickup elements which is included by the image pickup unit 11402 may be one (single-plate type) or a plural number (multi-plate type). Where the image pickup unit 11402 is configured as that of the multi-plate type, for example, image signals corresponding to respective R, G and B are generated by the image pickup elements, and the image signals may be synthesized to obtain a color image. The image pickup unit 11402 may also he configured so as to have a pair of image pickup elements for acquiring respective image signals for the right eye and the left eye ready for three dimensional (3D) display. If 3D display is performed, then the depth of a living body tissue in a surgical region can he comprehended more accurately by the surgeon 11131. It is to be noted that, where the image pickup unit 11402 is configured as that of stereoscopic type, a plurality of systems of lens units 11401 are provided corresponding to the individual image pickup elements.

Further, the image pickup unit 11402 may not necessarily be provided on the camera head 11102. For example, the image pickup unit 11402 may be provided immediately behind the objective lens in the inside of the lens barrel 11101.

The driving unit 11403 includes an actuator and moves the zoom lens and the focusing lens of the lens unit 11401 by a predetermined distance along an optical axis under the control of the camera head controlling unit 11405. Consequently, the magnification and the focal point of a picked up image by the image pickup unit 11402. can be adjusted suitably.

The communication unit 11404 includes a communication apparatus for transmitting and receiving various kinds of information to and from the CCU 11201. The communication unit 11404 transmits an image signal acquired from the image pickup unit 11402 as RAW data to the CCU 11201 through the transmission cable 11400.

In addition, the communication unit 11404 receives a control signal for controlling driving of the camera head 11102 from the CCU 11201 and supplies the control signal to the camera head controlling unit 11405. The control signal includes information relating to image pickup conditions such as, for example, information that a frame rate of a picked up image is designated, information that an exposure value upon image picking up is designated and/or information that a magnification and a focal point of a picked up image are designated.

It is to be noted that the image pickup conditions such as the frame rate, exposure value, magnification or focal point may be designated by the user or may be set automatically by the control unit 11413 of the CCU 11201 on the basis of an acquired image signal. In the latter case, an auto exposure (AE) function, an auto focus (AF) function and an auto white balance (AWB) function are incorporated in the endoscope 11100.

The camera head controlling unit 11405 controls driving of the camera head 11102 on the basis of a control signal from the CCU 11201 received through the communication unit 11404.

The communication unit 11411 includes a communication apparatus for transmitting and receiving various kinds of information to and from the camera head 11102. The communication unit 11411 receives an image signal transmitted thereto from the camera head 11102 through the transmission cable 11400.

Further, the communication unit 11411 transmits a control signal for controlling driving of the camera head 11102 to the camera head 11102. The image signal and the control signal can be transmitted by electrical communication, optical communication or the like.

The image processing unit 11412 performs various image processes for an image signal in the form of RAW data transmitted thereto from the camera head 11102.

The control unit 11413 performs various kinds of control relating to image picking up of a surgical region or the like by the endoscope 11100 and display of a picked up image obtained by image picking up of the surgical region or the like. For example, the control unit 11413 creates a control signal for controlling driving of the camera head 11102.

Further, the control unit 11413 controls, on the basis of an image signal for which image processes have been performed by the image processing unit 11412, the display apparatus 11202 to display a picked up image in which the surgical region or the like is imaged. Thereupon, the control unit 11413 may recognize various objects in the picked up image using various image recognition technologies. For example, the control unit 11413 can recognize a surgical tool such as forceps, a particular living body region, bleeding, mist when the energy device 11112 is used and so forth by detecting the shape, color and so forth of edges of objects included in a picked up image. The control unit 11413 may cause, when it controls the display apparatus 11202 to display a picked up image, various kinds of surgery supporting information to be displayed in an overlapping manner with an image of the surgical region using a result of the recognition. Where surgery supporting information is displayed in an overlapping manner and presented to the surgeon 11131, the burden on the surgeon 11131 can be reduced and the surgeon 11131 can proceed with the surgery with certainty.

The transmission cable 11400 which connects the camera head 11102 and the CCU 11201 to each other is an electric signal cable ready for communication of an electric signal, an optical fiber ready for optical communication or a composite cable ready for both of electrical and optical communications.

Here, while, in the example depicted, communication is performed by wired communication using the transmission cable 11400, the communication between the camera head 11102 and the CCU 11201 may be performed by wireless communication.

One example of the endoscopic surgery system to which the technology according to the present disclosure is applicable has been described above. The technology according to the present disclosure is applicable to, for example, the image pickup unit 11402 of the configurations described above. Applying the technology according to the present disclosure to the image pickup unit 11402 makes it possible to improve accuracy of an inspection.

It is to be noted that the endoscopic surgery system has been described here as an example, but the technology according to the present disclosure may be additionally applied to, for example, a microscopic surgery system and the like.

### Application Example 5

### <Example of Application to Mobile Body>

The technology according to the present disclosure is applicable to various products. For example, the technology according to the present disclosure may be achieved in the form of an apparatus to be mounted to a mobile body of any kind such as an automobile, an electric vehicle, a hybrid electric vehicle, a motorcycle, a bicycle, a personal mobility, an airplane, a drone, a vessel, a robot, a construction machine, and an agricultural machine (tractor).

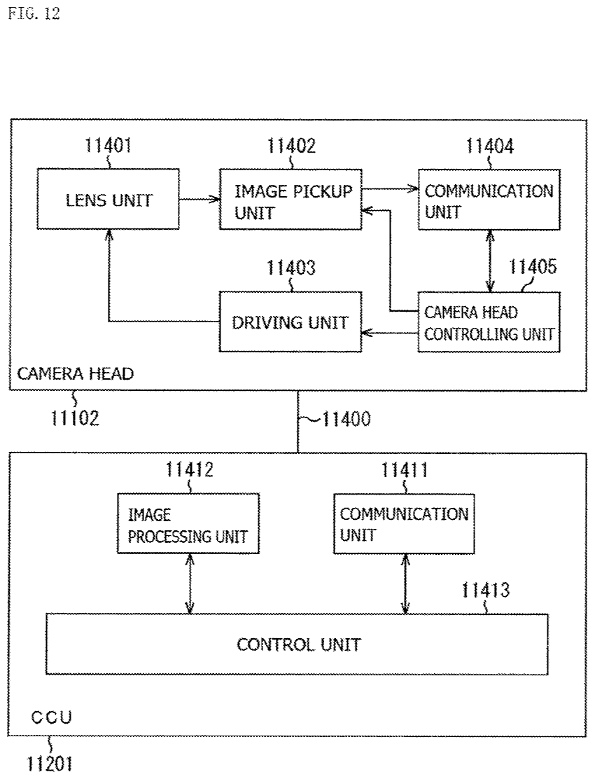

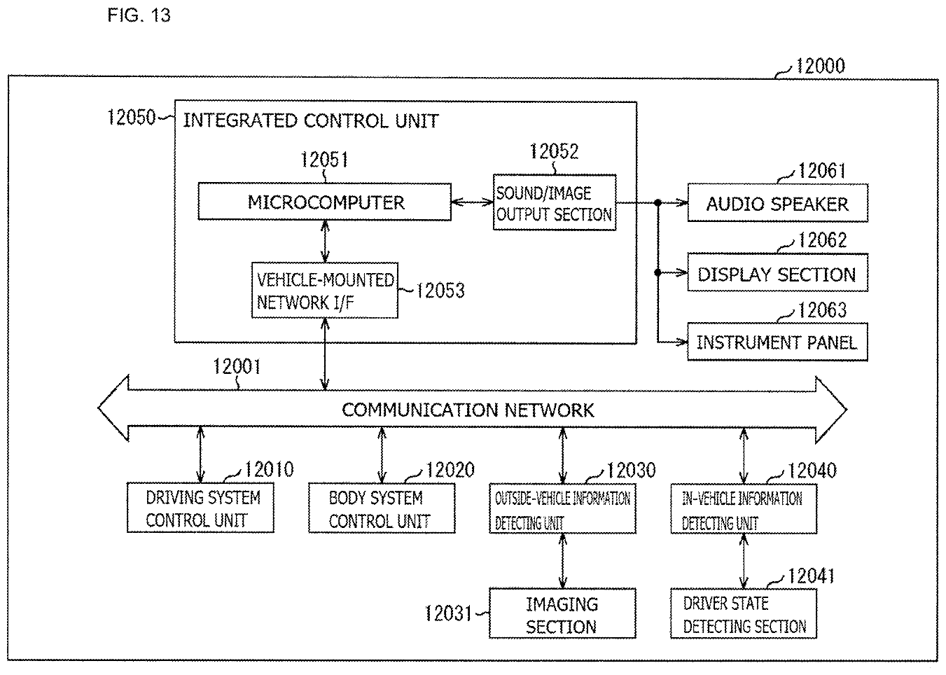

FIG. 13 is a block diagram depicting an example of schematic configuration of a vehicle control system as an example of a mobile body control system to which the technology according to an embodiment of the present disclosure can be applied.

The vehicle control system 12000 includes a plurality of electronic control units connected to each other via a communication network 12001. In the example depicted in FIG. 13, the vehicle control system 12000 includes a driving system control unit 12010, a body system control unit 12020, an outside-vehicle information detecting unit 12030, an in-vehicle information detecting unit 12040, and an integrated control unit 12050. In addition, a microcomputer 12051, a sound/image output section 12052, and a vehicle-mounted network interface (I/F) 12053 are illustrated as a functional configuration of the integrated control unit 12050.

The driving system control unit 12010 controls the operation of devices related to the driving system of the vehicle in accordance with various kinds of programs. For example, the driving system control unit 12010 functions as a control device for a driving force generating device for generating the driving force of the vehicle, such as an internal combustion engine, a driving motor, or the like, a driving force transmitting mechanism for transmitting the driving force to wheels, a steering mechanism for adjusting the steering angle of the vehicle, a braking device for generating the braking force of the vehicle, and the like.

The body system control unit 12020 controls the operation of various kinds of devices provided to a vehicle body in accordance with various kinds of programs. For example, the body system control unit 12020 functions as a control device for a keyless entry system, a smart key system, a power window device, or various kinds of lamps such as a headlamp, a backup lamp, a brake lamp, a turn signal, a fog lamp, or the like. In this case, radio waves transmitted from a mobile device as an alternative to a key or signals of various kinds of switches can be input to the body system control unit 12020. The body system control unit 17)020 receives these input radio waves or signals, and controls a door lock device, the power window device, the lamps, or the like of the vehicle.

The outside-vehicle information detecting unit 12030 detects information about the outside of the vehicle including the vehicle control system 12000. For example, the outside-vehicle information detecting unit 12030 is connected with an imaging section 12031. The outside-vehicle information detecting unit 12030 makes the imaging section 12031 image an image of the outside of the vehicle, and receives the imaged image. On the basis of the received image, the outside-vehicle information detecting unit 12030 may perform processing of detecting an object such as a human, a vehicle, an obstacle, a sign, a character on a road surface, or the like, or processing of detecting a distance thereto.

The imaging section 12031 is an optical sensor that receives light, and which outputs an electric signal corresponding to a received light amount of the light. The imaging section 12031 can output the electric signal as an image, or can output the electric signal as information about a measured distance. In addition the light received by the imaging section 12031 may be visible light, or may be invisible light such as infrared rays or the like.

The in-vehicle information detecting unit 12040 detects information about the inside of the vehicle. The in-vehicle information detecting unit 12040 is, for example, connected with a driver state detecting section 12041 that detects the state of a driver The driver state detecting section 12041, for example, includes a camera that images the driver. On the basis of detection information input from the driver state detecting section 12041, the in-vehicle information detecting unit 12040 may calculate a degree of fatigue of the driver or a degree of concentration of the driver, or may determine whether the driver is dozing.

The microcomputer 12051 can calculate a control target value for the driving force generating device, the steering mechanism, or the braking device on the basis of the information about the inside or outside of the vehicle which information is obtained by the outside-vehicle information detecting unit 12030 or the in-vehicle information detecting unit 12040, and output a control command to the driving system control unit 12010. For example, the microcomputer 12051 can perform cooperative control intended to implement functions of an advanced driver assistance system (ADAS) which functions include collision avoidance or shock mitigation for the vehicle, following driving based on a following, distance, vehicle speed maintaining driving, a warning of collision of the vehicle, a warning of deviation of the vehicle from a lane, or the like.

In addition, the microcomputer 12051 can perform cooperative control intended for automatic driving, which makes the vehicle to travel autonomously without depending on the operation of the driver, or the like, by controlling the driving force generating device, the steering mechanism, the braking device, or the like on the basis of the information about the outside or inside of the vehicle which information is obtained by the outside-vehicle information detecting unit 12030 or the in-vehicle information detecting unit 12040.

In addition, the microcomputer 12051 can output a control command to the body system control unit 12020 on the basis of the information about the outside of the vehicle which information is obtained by the outside-vehicle information detecting unit 12030. For example, the microcomputer 12051 can perform cooperative control intended to prevent a glare by controlling the headlamp so as to change from a high beam to a low beam, for example, in accordance with the position of a preceding vehicle or an oncoming vehicle detected by the outside-vehicle information detecting unit 12030.

The sound/image output section 12052 transmits an output signal of at least one of a sound and an image to an output device capable of visually or auditorily notifying information to an occupant of the vehicle or the outside of the vehicle. In the example of FIG. 13, an audio speaker 12061, a display section 12062, and an instrument panel 12063 are illustrated as the output device. The display section 12062 may, for example, include at least one of an on-board display and a head-up display.

FIG. 14 is a diagram depicting an example of the installation position of the imaging section 12031.

In. FIG. 14, the imaging section 12031 includes imaging sections 12101, 12102, 12103, 12104, and 12105.

The imaging sections 12101, 12102, 12103, 12104, and 12105 are, for example, disposed at positions on a front nose, sideview mirrors, a rear bumper, and a back door of the vehicle 12100 as well as a position on an upper portion of a windshield within the interior of the vehicle. The imaging section 12101 provided to the front nose aid the imaging section 12105 provided to the upper portion of the windshield within the interior of the vehicle obtain mainly an image of the front of the vehicle 12100. The imaging sections 12102 and 12103 provided to the sideview mirrors obtain mainly an image of the sides of the vehicle 12100. The imaging section 12104 provided to the rear bumper or the back door obtains mainly an image of the rear of the vehicle 12100. The imaging section 12105 provided to the upper portion of the windshield within the interior of the vehicle is used mainly to detect a preceding vehicle, a pedestrian, an obstacle, a signal, a traffic sign, a lane, or the like.

Incidentally, FIG. 14 depicts an example of photographing ranges of the imaging sections 12101 to 12104. An imaging range 12111 represents the imaging range of the imaging section 12101 provided to the front nose. Imaging ranges 12112 and 12113 respectively represent the imaging ranges of the imaging sections 12102 and 12103 provided to the sideview mirrors. An imaging range 12114 represents the imaging range of the imaging section 12104 provided to the rear bumper or the back door. A bird's-eye image of the vehicle 12100 as viewed from above is obtained by superimposing image data imaged by the imaging sections 12101 to 12104, for example.

At least one of the imaging sections 12101 to 12104 may have a function of obtaining distance information. For example, at least one of the imaging sections 12101 to 12104 may be a stereo camera constituted of a plurality of imaging elements, or may be an imaging element having pixels for phase difference detection.

For example, the microcomputer 12051 can determine a distance to each. three-dimensional object within the imaging ranges 12111 to 12114 and a temporal change in the distance (relative speed with respect to the vehicle 12100) on the basis of the distance information obtained from the imaging sections 12101 to 12104, and thereby extract, as a preceding vehicle, a nearest three-dimensional object in particular that is present on a traveling path of the vehicle 12100 and which travels in substantially the same direction as the vehicle 12100 at a predetermined speed (for example, equal to or more than 0 km/hour), Further, the microcomputer 12051 can set a following distance to be maintained in front of a preceding vehicle in advance, and perform automatic brake control (including following stop control), automatic acceleration control (including following start control), or the like. It is thus possible to perform cooperative control intended for automatic driving that makes the vehicle travel autonomously without depending on the operation of the driver or the like.

For example, the microcomputer 12051 can classify three-dimensional object data on three-dimensional objects into three-dimensional object data of a two-wheeled vehicle, a standard-sized vehicle, a large-sized vehicle, a pedestrian, a utility pole, and other three-dimensional objects on the basis of the distance information obtained from the imaging sections 12101 to 12104, extract the classified three-dimensional object data, and use the extracted three-dimensional object data for automatic avoidance of an obstacle. For example, the microcomputer 12051 identifies obstacles around the vehicle 12100 as obstacles that the driver of the vehicle 12100 can recognize visually and obstacles that are difficult for the driver of the vehicle 12100 to recognize visually Then, the microcomputer 12051 determines a collision risk indicating a risk of collision with each obstacle. In a situation in which the collision risk is equal to or higher than a set value and there is thus a possibility of collision, the microcomputer 12051 outputs a warning to the driver via the audio speaker 12061 or the display section 12062, and performs forced deceleration or avoidance steering via the driving system control unit 12010. The microcomputer 12051 can thereby assist in driving to avoid collision.

At least one of the imaging sections 12101 to 12104 may be an infrared camera that detects infrared rays. The microcomputer 12051 can, for example, recognize a pedestrian by determining Whether or not there is a pedestrian in imaged images of the imaging sections 12101 to 12104. Such recognition of a pedestrian is, for example, performed by a procedure of extracting characteristic points in the imaged images of the imaging sections 12101 to 12104 as infrared cameras and a procedure of determining whether or not it is the pedestrian by performing pattern matching processing on a series of characteristic points representing the contour of the object. When the microcomputer 12051 determines that there is a pedestrian in the imaged images of the imaging sections 12101 to 12104, and thus recognizes the pedestrian the sound/image output section 12052 controls the display section 12062 so that a square contour hue for emphasis is displayed so as to be superimposed on the recognized pedestrian. The sound image output section 12052 may also control the display section 12062 so that an icon or the like representing the pedestrian is displayed at a desired position.

### 4. EXAMPLES

Next, examples of the present disclosure are described in detail below.

### [Experiment 1]

### (Fabrication of Element for Evaluation)

First, as a material used for an organic photoelectric conversion layer, a BBBT derivative (BBBT-1) represented by an expression (5) Was synthesized by the following synthesis scheme (Chem 7). In addition, as the material used for the organic photoelectric conversion layer, a BBBT derivative (BBBT-2) represented by the above-described expression (1-1) was synthesized by the following synthesis scheme (Chem. 8). Each of thus-obtained crude compounds BBBT-1 and BBBT-2 was sublimed and refined.

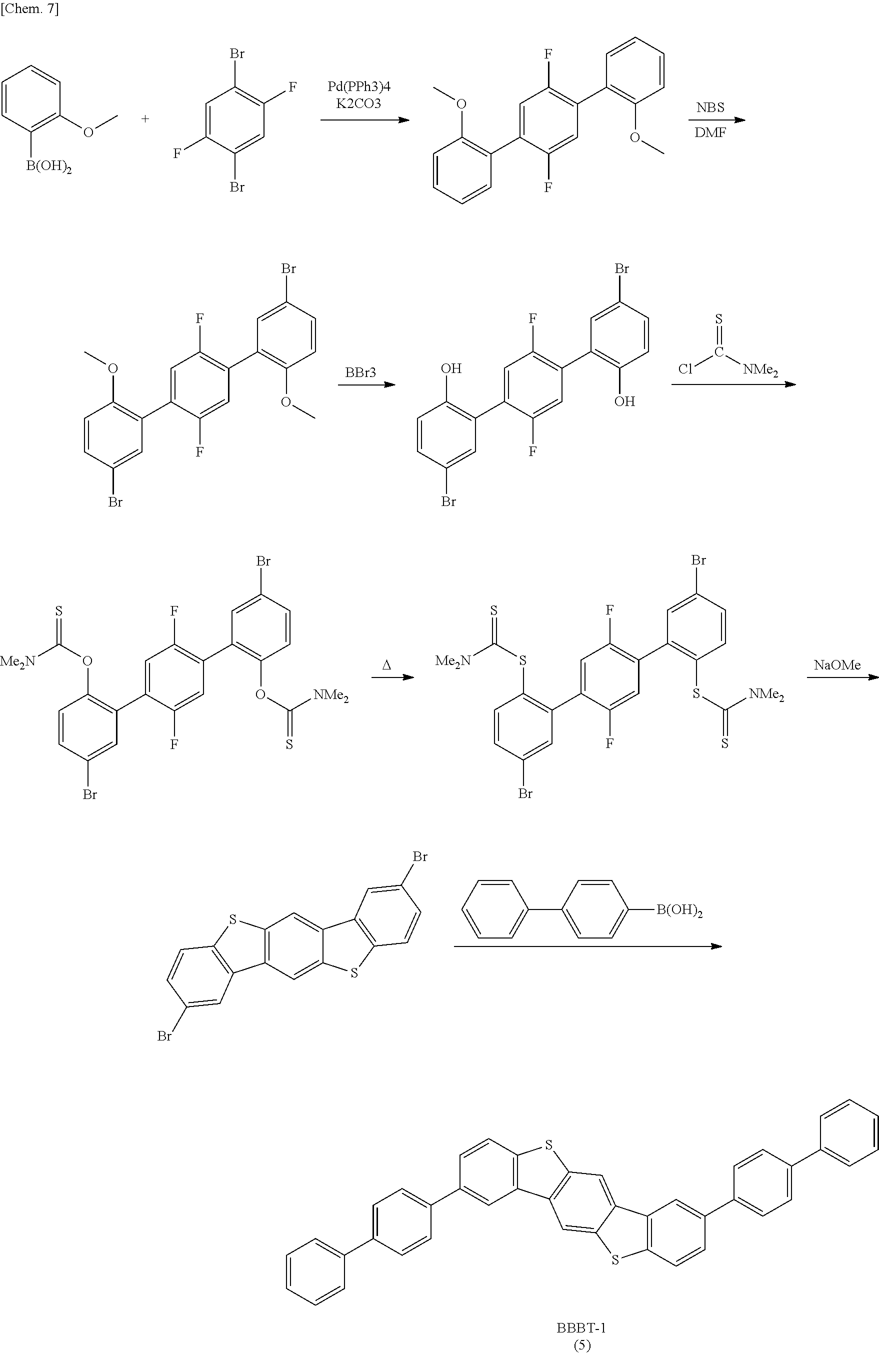

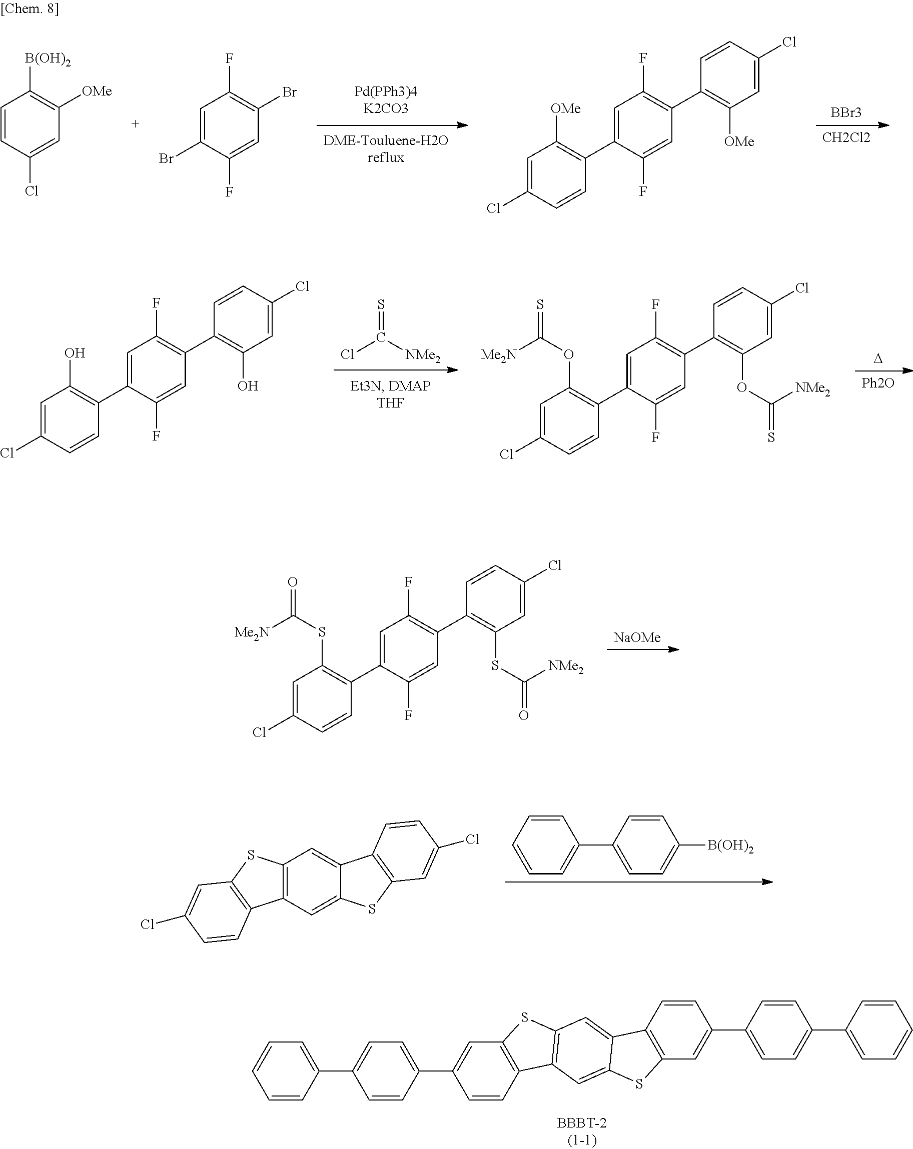

### Experimental Example 1

Subsequently, a photoelectric conversion element haying a cross-sectional configuration illustrated in FIG. 15 was fabricated with use of the compound BBBT-1 by the following method. First, an ITO film having a thickness of 120 nm was formed on a quartz substrate 111 by a sputtering apparatus, and thereafter, a lower electrode 112 was formed by patterning with use of a lithography technology using a photomask. Subsequently, an insulating layer 113 was formed on the quartz substrate 111 and the lower electrode 112, and an opening from which the lower electrode 112 of 1 mm square was exposed was formed with use of a lithography technology, followed by ultrasonic cleaning sequentially with a neutral detergent, acetone, and ethanol. The quartz substrate 111 was dried, and thereafter, an UV/ozone (O₃) treatment was performed for 10 minutes. Subsequently, the compound BBBT-1, fluorinated subphthalocyanine chloride (F₆-SubPc-OC₆F₅) represented by the following expression (4-1), and C60 fullerene represented by the following expression (2-1) were co-evaporated at an evaporation speed ratio of 4:4:2 in vacuum evaporation film formation using a shadow mask to form the organic photoelectric conversion layer 114 having a thickness of 230 nm. Subsequently, a film of B4PyMPM. represented by the following expression (6) was formed as a buffer layer 115 to have a thickness of 5 nm. Next, a film of an Al—Si—Cu alloy was formed as the upper electrode 116 on the buffer layer 115 by evaporation to have a thickness of 100 nm, and thereafter, annealing was performed at 160° C. for five minutes in a nitrogen atmosphere. Thus, the photoelectric conversion element (experimental example 1) was fabricated.

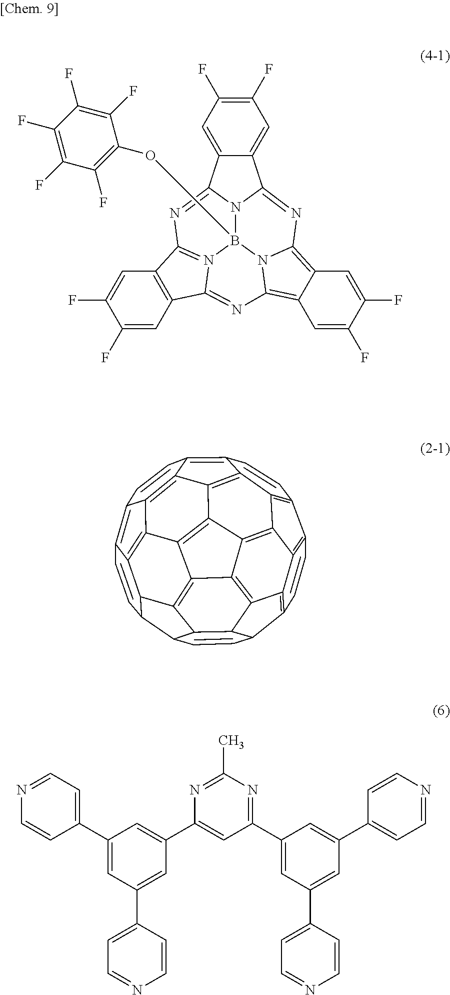

### Experimental Example 2

Next, a photoelectric conversion element (experimental example 2) was fabricated by a method similar to that in the experimental example 1, except that the compound BBBT-2 was used instead of the compound BBBT-1.

### (Physical Property Evaluation of Materials Used for Organic Photoelectric Conversion Layer)

Energy evaluation of the materials (the compound BBBT-1 and the compound BBBT-2) used for the organic photoelectric conversion layer was performed by the following method. First, a thin film of each of the compound BBBT-1 and the BBBT-2 having a thickness of 20 nm was formed on a Si substrate, and a surface of the thin film was measured by ultraviolet photoelectron spectroscopy (UPS) to determine a HOMO level (ionization potential). An optical energy gap was calculated from an absorption end of an absorption spectrum of each of the thin films of the compound BBBT-1 and the compound BBBT-2, and a LUMO (Lowest Unoccupied Molecular Orbital) level was calculated from a difference between the energy gap and the HOMO level (LUMO=−1*||HOMO|−energy gap|).

The photoelectric conversion elements (the experimental example 1 and the experimental example 2) were evaluated with use of the following method. First, each of the photoelectric conversion elements was placed on a prober stage, and while a voltage of −1 V (a so-called reverse bias voltage of 1 V) was applied between the lower electrode and the upper electrode, each of the photoelectric conversion elements was irradiated with light on conditions of a wavelength of 360 nm and 2 μW/cm² to measure a light current. Thereafter, light irradiation was stopped, and a dark current was measured. Next, in accordance with the following expression, external quantum efficiency (EQE=|((light current−dark current)×100/(2×10\hat{ }−6))×(1240/560)×100|) was determined from the light current and the &Irk current.

TABLE 1

| | BBBT-1 | BBBT-2 |
|---|---|---|
| HOMO (eV) | −5.7 | −5.8 |
| LUMO (eV) | −2.6 | −2.9 |
| EQE (Relative Value) | 1.00 | 16.6 |
| Dark Current | 1.00 | 1.01 |
| (Relative Value) | | |

Table 1 is a summary of the HOMO levels and LUMO levels of the materials (the compound BBBT-1 and the compound BBBT-2) used for the organic photoelectric conversion layer, and EQE (a relative value) and the dark currents (a relative value) of the photoelectric conversion elements (the experimental example 1 and the experimental example 2) formed using these materials. From Table 1, the photoelectric: conversion element (the experimental example 2) using the compound BBBT-2 Obtained EQE about 17 times greater than that in the photoelectric conversion element (the experimental example 1) using the compound BBBT-1. There was no difference in the dark current value between the materials.

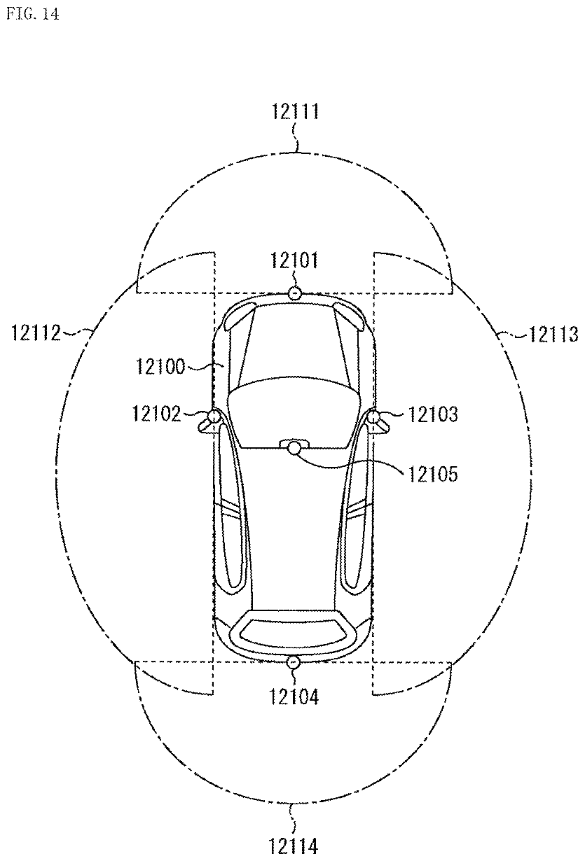

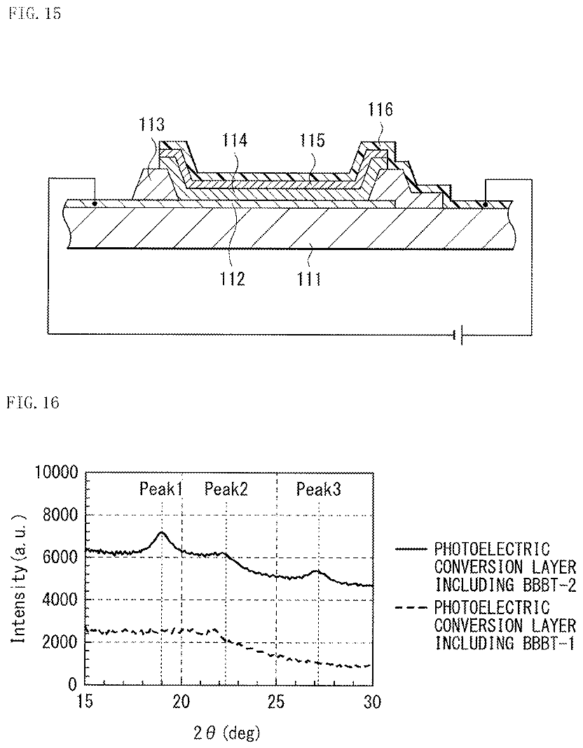

In order to consider a difference ire EQE between the experimental example 1 using the compound BBBT-1 and the experimental example 2 using the compound BBBT-2, organic photoelectric conversion layers having a similar configuration were separately fabricated, and XRD measurement was performed. FIG. 16 illustrates results of the measurement. In the organic: photoelectric conversion layer including the compound BBBT-2, three apparent peaks were confirmed. In contrast, the organic photoelectric conversion layer including the compound BBBT-1 showed a broad XRD chart. Further, a single-layer film of each of the compound BBBT-1 and the compound BBBT-2 was fabricated, and XRD measurement was performed. FIG. 17 illustrates results of the measurement. Even in a case where the measurement was performed on the single-layer film of the compound BBBT-2, three apparent peaks were confirmed. That is, it was found out that even if a subphthalocyanine compound and fullerene were mixed in addition to the compound BBBT-2 to form the organic photoelectric conversion layer, orientation formed by the compound BBBT-2 was maintained. In contrast, as for the compound BBBT-1, it was possible to confirm only one apparent peak in the single-layer film, but in the organic photoelectric conversion layer, the apparent peak disappeared, and a broad XRD chart was shown. That is, it was found out that even if the compound BBBT-1 was used as a single layer, crystallinity was low, and in a case where the compound BBBT-1 was used as the material of the organic photoelectric conversion layer together with another material, the crystallinity was further decreased.

Next, X-ray structural analysis of powder of the compound BBBT-1 and the compound BBBT-2 was also executed. In the compound BBBT-1, a stacking state of BBBT mother skeletons was misaligned in a long axis direction. Further, it appeared that affinity called CH/π interaction exerted between carbon and hydrogen of another compound BBBT-1 molecule and it electrons of a BBBT mother skeleton was not exerted so much. That is, it was suggested that the BBBT derivative had a high possibility that crystallization is impaired by a position of a substituent group.

In contrast, the compound BBBT-2 is a linear molecule including a substituent group, and it is considered that an interaction with another molecule is not impaired by the substituent group. In addition, in the compound BBBT-2, it is presumable from an XRD chart of a thin film that three kinds of orientation are possible and it is presumed that a three-dimensional carrier path is formed both in a single-layer film and in the organic photoelectric conversion layer.

As described above, it is considered that, in the BBBT derivative, a molecular orientation property, and by extension to crystallinity and a grain size are greatly changed by the position of the substituent group provided to the BBBT mother skeleton. Accordingly, as illustrated in Table 1, it is considered that a large difference ire EQE was caused between the respective photoelectric conversion elements (the experimental example 1 and the experimental example 2) using the compound BBBT-1 and the compound BBBT-2.

### [Experiment 2]

### (Fabrication of Elements for Evaluation)

First, as a material used for an organic photoelectric conversion layer, a compound BP-rBDT represented by an expression (7) was synthesized by the following synthesis scheme (Chem. 10). A thus-obtained crude compound BP-rBDT was sublimed and refined.

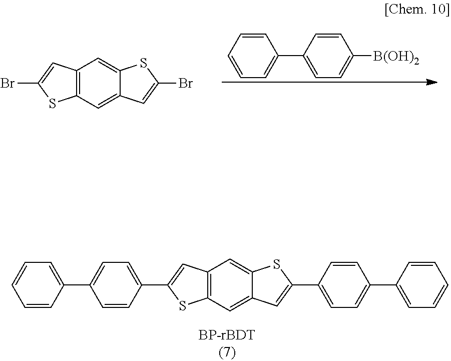 [SMILES: Brc1cc2cc3sc(Br)cc3cc2s1 ; OB(O)c1ccc(-c2ccccc2)cc1 ; c1ccc(-c2ccc(-c3cc4cc5sc(-c6ccc(-c7ccccc7)cc6)cc5cc4s3)cc2)cc1]

### Experimental Example 3

A photoelectric conversion element was fabricated with use of the compound BP-rBDT by the following Method. First, an ITO film having a thickness of 120 nm was formed on a silicon substrate by a sputtering apparatus, and thereafter, a lower electrode was formed by patterning with use of a lithography technology using a photomask. Subsequently, an insulating layer was formed on the silicon substrate and the lower electrode, and an opening from which the lower electrode of 1 mm square was exposed was firmed with use of a lithography technology, followed by ultrasonic cleaning sequentially with a neutral detergent, acetone, and ethanol. The silicon substrate seas dried, and thereafter, an UV/ozone (O₃) treatment was performed for 10 minutes. Thereafter, the silicon substrate was fixed to a substrate holder of an evaporation apparatus, and thereafter, an evaporation layer was decompressed to 5.5×10⁻⁵ Pa. Subsequently a film of an indolocarbazole derivative represented by the following expression (8) was formed as a buffer layer in vacuum evaporation film formation using, a shadow mask to have a thickness of 10 nm. Subsequently, the compound BP-rBDT, fluorinated subphthalocyanine chloride (F₆-SubPc-OC₆F₅) represented by the following expression (4-1), and C60 fullerene represented by the following expression (2-1) were co-evaporated at an evaporation speed ratio of 4:4:2 to form an organic photoelectric conversion layer having a thickness of 230 nm. Subsequently, a film of B4PyMPM represented by the above-described expression (6) was formed as a buffer layer to have a thickness of 5 nm. Then, the buffer layer was placed in a container that was transportable in an inert atmosphere, was transported to a sputtering apparatus, and a film of ITO having a thickness of 50 nm was formed as an upper electrode on the buffer layer. Thereafter, in a nitrogen atmosphere, annealing simulating a heating process such as soldering of an element was performed at 150° C. for 3.5 h to fabricate a photoelectric conversion element (experimental example 3).

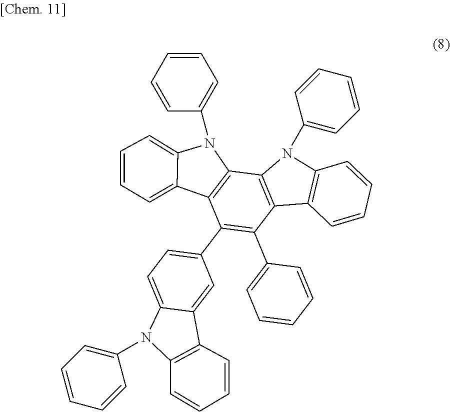 [SMILES: c1ccc(-c2c(-c3ccc4c(c3)c3ccccc3n4-c3ccccc3)c3c4ccccc4n(-c4ccccc4)c3c3c2c2ccccc2n3-c2ccccc2)cc1]

### Experimental Example 4

Next, a photoelectric conversion element (experimental example 4) was fabricated by a method similar to that in the experimental example 3, except that the compound BBBT-2 was used instead of the compound BP-rBDT.

### (Physical Property Evaluation of Materials Used for Organic Photoelectric Conversion Layer)

Energy evaluation of the materials (the compound BP-rBDT and the compound BBBT-2) used for the organic photoelectric conversion layer was performed by a method similar to that in the above-described experiment 1.

As for mobility, an element for hole mobility measurement was fabricated, and mobility thereof was evaluated by the following method. First, a thin film of platinum (Pt) having a thickness of 100 nm was formed by an EB evaporation method, and a platinum electrode was formed on the basis of a lithography technology using a photomask. Next, an insulating layer was formed on the Substrate and the platinum electrode, and pixels were formed to cause the platinum electrode of 0.25 mm square to be exposed by a lithography technology. Then, a molybdenum oxide (MoO₃) film having 1 nm, films of the compounds BP-rBDT and the compound BBBT-2, of which mobility was to be measured, having 200 nm, a molybdenum oxide (MoO₃) film having 3 nm, and a gold electrode having 100 nm each were formed and stacked. A voltage of −1 V to −20 V or a voltage of +1 V to +20 V was applied to the thus-obtained element for mobility evaluation, an expression of SCLC (pace charge limited current) was fitted to a current-voltage curve where more current flowed by a negative bias or a positive bias, and mobility at −1 V or +1 measured,

The photoelectric conversion elements (the experimental example 3 and the experimental example 4) were evaluated by the following method. First, each of the photoelectric conversion elements was placed on a prober stage previously warmed at 60° C., and while a voltage of −2.6 V (a so-called reverse bias voltage of 2.6 V) was applied between the lower electrode and the upper electrode, each of the photoelectric conversion elements was irradiated with light on conditions of a wavelength of 560 nm and 2 μW/cm² to measure a light current. Thereafter, light irradiation was stopped and a dark current was measured. Next, in accordance with the following expression, external quantum efficiency (EQE=|((light current−dark current)×100/(2×10\hat{ }−6))×(1240/560)×100|) was determined from the light current and the dark current. In addition, as for afterimage evaluation, each of the photoelectric conversion elements was irradiated with light on conditions of a wavelength of 560 nm and 2 μW/cm² while applying −2.6 V between the lower electrode and the upper electrode, and subsequently, when light irradiation was stopped, the amount of current flowing between the second electrode and the first electrode immediately before the light irradiation was stopped was I₀ and time (T₀) from the stop of the light irradiation until the current amount reached (0.03×I₀) was afterimage time.

TABLE 2

| | BP-rBDT | BBBT-2 |
|---|---|---|
| HOMO (eV) | −5.6 | −5.8 |
| LUMO (eV) | −2.8 | −2.9 |
| Apparent HOMO (eV) | −5.6 | −6.1 |
| Hole Mobility (cm²/Vs) | 8.60E−06 | 2.20E−05 |
| EQE (Relative Value) | 1.00 | 0.99 |
| Dark Current | 1.00 | 0.01 |
| (Relative Value) | | |
| Afterimage | 1.00 | 0.67 |
| Characteristics | | |
| (Relative Value) | | |

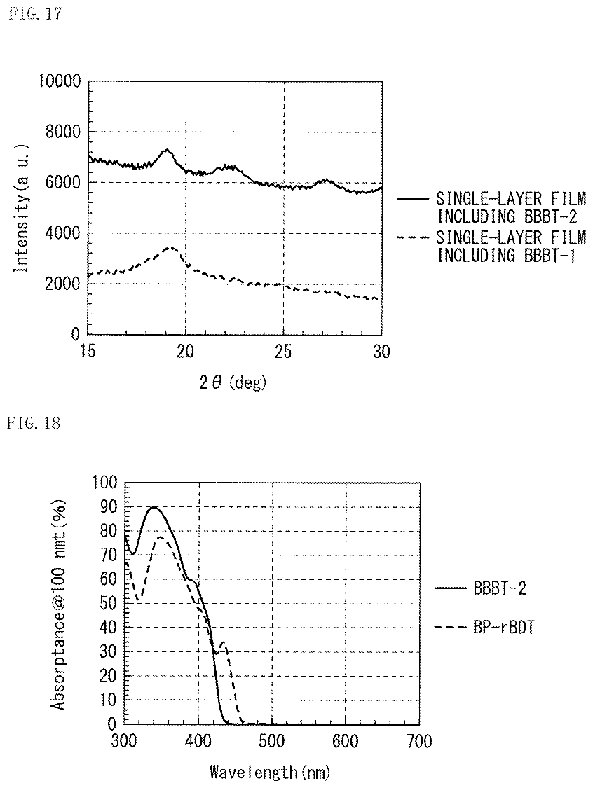

Table 2 is a summary of the HOMO levels, and LUMO levels, apparent HOMO levels, and hole mobility of the materials (the compound BP-rBDT and the compound BBBT-2) used for the organic photoelectric conversion layer, and EQE (a relative value), the dark currents (a relative value), and afterimage characteristics (a relative value) of the photoelectric conversion elements (the experimental example 3 and the experimental example 4) formed using these materials. FIG. 18 illustrates absorption spectra of the compound BP-rBDT and the compound BBBT-2 in a case where films having a film thickness of 50 nm of the compound BP-rBDT and the compound BBBT-2 were formed on quartz substrates by evaporation and the film thickness was converted into a film thickness of 100 nm. The compound BBBT-2 had smaller absorption of visible light, as compound with the compound BP-rBDT. This provides characteristics that photoelectric conversion of only a desired wavelength region is selectively performed in a case where the compound BBBT-2 is used as the organic photoelectric conversion layer or the buffer layer. Further, in a case where this photoelectric conversion element is used in a stacked-type imaging element, an effect of preventing interference with photoelectric conversion is exerted on an element provided below an element including the BBBT derivative with respect to a light incident direction. In addition, spectral characteristics of the compound BBBT-2 was favorable, as compared with a typical organic semiconductor.

In addition, it was found out from Table 2 that the compound BBBT-2 had EQE substantially equal to that of the compound BP-rBDT but the dark current was suppressed to one-hundredth of the dark current of the compound PB-rBDT. In addition, it was found out that it was possible to reduce the afterimage characteristics to two-third. It is considered that this is caused by a difference between molecular structures of the compound BBBT-2 and the compound BP-rBDT.

The difference between molecular structures of the compound BBBT-2 and the compound BP-rBDT is in the number of rings of the mother skeleton. It is considered that, as for the dark current, it is because delocalization energy of π electrons in the mother skeleton is increased with an increase in the number of rings of the mother skeleton, resulting in a decrease in the HOMO level. As illustrated in Table 2, an actually measured value of the HOMO level of the compound BBBT-2 was deeper by 0.2 eV than that of the compound BP-rBDT.

FIG. 19 illustrates vacuum levels of the compound BP-rBDT, the compound BBBT-2, fluorinated subphthalocyanine chloride (F₆-SubPcOC₆F₅), and C60 fullerene in the organic photoelectric conversion layer (i layer), The HOMO levels of the compound BBBT-2 and the compound BP-rBDT in the organic photoelectric conversion layer vary by an influence of a subphthalocyanine derivative and C60 fullerene in the organic photoelectric conversion layer. Accordingly, in a case where apparent HOMO levels of the compound BBBT-2 and the compound BP-rBDT in the organic photoelectric conversion layer were measured, the HOMO level of the compound BP-rBDT had a value substantially equal to that in a case of a single-layer film of the compound BP-rBDT, but the HOMO level of the compound BBBT-2 became −6.1 eV that was deeper. This means that an energy difference (AP) between the LUMO level of the subphthalocyanine derivative or C60 fullerene in the organic photoelectric conversion layer and the HOMO level of the compound BBBT-2 was further increased, and it is considered that carrier movement at the dark time was suppressed, as compared with the compound BP-rBDT. Accordingly, it was found out that an energy difference (ΔE) between the HOMO level of the organic semiconductor represented by a compound (1) and the LUMO level of a material other than the compound (1) in the photoelectric conversion layer was preferably larger than 1.1 eV and more preferably larger than 1.6 eV.

In addition, in linear molecules such as the compound BBBT-2 and the compound BP-rBDT, the number of condensed rings in a benzene ring is increased to decrease a ratio of different kinds of elements in a mother skeleton, thereby moderately relaxing intermolecular interaction to make a grain size formed by the BBBT derivative moderate. In a case where the grain size was too large, a contact property between gains was decreased, and a dense film was not formed. In a case of a moderate grain size, the contact property between grains is favorable; therefore, it is considered that carrier transportability between grains is improved and mobility of the thin film is improved.

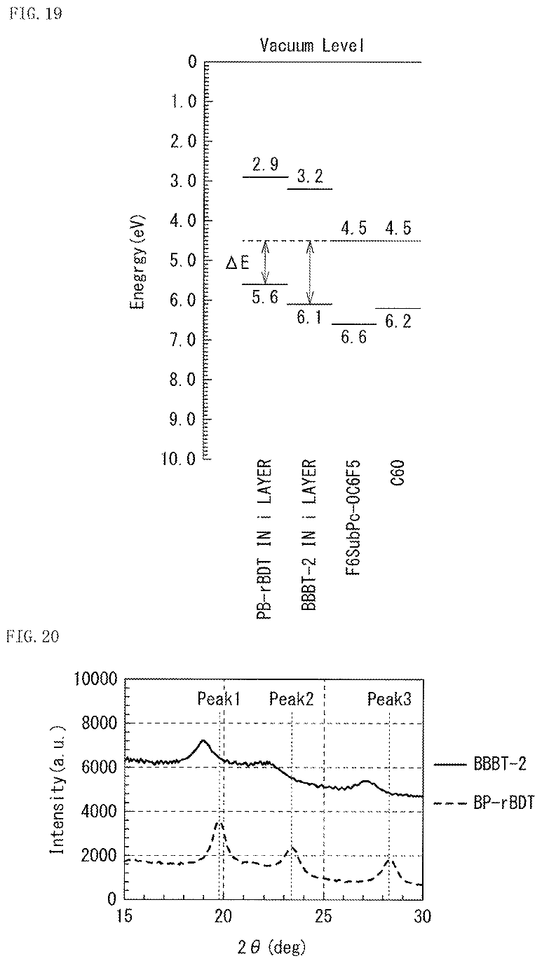

In order to confirm this, organic photoelectric conversion layers having configurations similar to those in the experimental example 3 using the compound BP-rBDT and the experimental example 4 using the compound BBBT-2 were separately fabricated, and XRD measurement was performed. FIG. 20 illustrates results of the measurement, and Table 3 illustrates respective particle diameters at three peak positions of the compound BP-rBDT and the compound BBBT-2. All three peaks of the compound BBBTA were shifted to a low angle side, as compared with the compound. BP-rBDT. This indicates that the compound BBBT-2 has larger crystal lattice spacing than the compound BP-rBDT. That is, the compound BBBT-2 is considered to have smaller intermolecular interaction than the compound BP-rBDT. Actually, in a case where particle diameters at three peaks illustrated in FIG. 20 were calculated with use of the Scherrer's equation, the particle diameters of the BBBT-2 were smaller than those of BP-rBDT. It is possible to construe from those that BBBT-2 had a low cohesive property; therefore, a denser film was formed and favorable mobility was obtained. Actually, as illustrated in Table 2, hole mobility of the compound BBBT-2 having two more rings than BP-rBDT had a value one order of magnitude greater than that of the compound BP-rBDT. It is presumable that this is a factor causing a decrease in afterimage characteristics of the compound BBBT-2 to about one-third of those of the compound BP-rBDT. Further, it is considered that a moderate gain size formed by the BBBT derivative reduces traps existing between. crystal grains, and it is assumed that this leads to favorable dark current characteristics.

TABLE 3

Particle Diameter (nm)

| | BP-rBDT | BBBT-2 |
|---|---|---|
| Peak1 | 12.87 | 6.66 |
| Peak2 | 11.29 | 7.14 |
| Peak3 | 11.32 | 7.93 |

As described above, the BBBT mother skeleton is considered as a superior material that exhibits favorable photoelectric conversion characteristics by linearly substituting a substituent group. Moreover, as can be seen from the results of the experiment 1 and the experiment 2, using the benzobisbenzothiophene (BBBT) derivative represented by the above-described general expression (1) for the photoelectric conversion element, the stacked-type imaging element, and the like makes it possible to achieve superior dark current characteristics and superior afterimage characteristics in addition to favorable photoelectric conversion efficiency.

Although the description has been given by referring to the embodiment, the modification examples 1 and 2, and the examples, the contents of the present disclosure are not limited to the above-described embodiment and the like, and may be modified in a variety of ways. For example, in the above-described embodiment, the photoelectric conversion element has a configuration in which the organic photoelectric converter 11G detecting green light and the inorganic photoelectric converters 11B and 11R respectively detecting blue light and red light are stacked; however, the contents of the present disclosure is not limited to such a configuration. That is, the organic photoelectric converter may detect red light or blue light, and the inorganic photoelectric converter may detect green light.

In addition, in the modification example 1 and FIG. 6, an example in which the red photoelectric: converter 40R, the green photoelectric converter 40G, and the blue photoelectric converter 40B are stacked in this order on the silicon substrate 81 has been described, but this is not limitative. For example, the green photoelectric converter 40G may be disposed on the light incident surface side by replacing the green photoelectric converter 40G and the blue photoelectric converter 40B with each other.

Further, the Amber of organic photoelectric converters, the number of inorganic photoelectric converters, a ratio between the organic photoelectric converters and the inorganic photoelectric converters are not limited, and two or more organic photoelectric converters may be provided, or color signals of a plurality of colors may be acquired only by the organic photoelectric converter, as described in the modification example 1. In this case, examples of arrangement of the respective organic photoelectric converters may include not only a longitudinal spectral type and a Bayer arrangement, but also an interline arrangement, a G stripe RB checkered arrangement, a G stripe RB complete checkered arrangement, a checkered complementary color arrangement, a stripe arrangement, a diagonal stripe arrangement, a primary-color color difference arrangement, a field color difference sequential arrangement, a frame color difference sequential arrangement, a MOS-type arrangement, an improved MOS-type arrangement, a flame interleave arrangement, and a field interleave arrangement. Furthermore, the content of the present disclosure is not limited to a configuration in which organic photoelectric converters and inorganic photoelectric converters are stacked in the longitudinal direction, and organic photoelectric converters and inorganic: photoelectric converters may be arranged side by side along a substrate surface.

Further, in the modification example 1, the configuration of the longitudinal spectral system imaging element in which the red photoelectric converter 40R, the green photoelectric converter 40G, and the blue photoelectric converter 40B are stacked on the silicon substrate 81 with the insulating layer 82 interposed therebetween has been described; but this is not limitative. For example, an imaging element may have a so-called Bayer arrangement in which pixels of three colors having corresponding photoelectric converters (the red photoelectric converter 40R, the green photoelectric converter 40G, and the blue photoelectric converter 40B) are arranged in a plane. The imaging element having the Bayer arrangement makes it possible to relax specifications of spectral characteristics of the respective photoelectric converters 40R, 40G, and 40B, as compared with the longitudinal spectral system imaging element, which makes it possible to improve mass-productivity.

It is to be noted that, in a case where the red photoelectric converter 40R, the green photoelectric converter 40G, and the blue photoelectric converter 40B are :arranged side by side on the substrate as in the Bayer arrangement, one (an electrode on a side opposite to a light incident side) of a pair of electrodes included in each of the photoelectric converters 40R, 40G, and 40B does not necessarily have light transmissivity and may be formed using a metal material. Specific examples of the metal material include aluminum (Al), an Al—Si—Cu alloy, a Mg—Ag alloy, an Al—Nd alloy, ASC (an alloy of aluminum, samarium, and same), and the like.

In addition, in a case where it does not matter if electrodes included in the organic photoelectric converter 11G, the red photoelectric converter 40R, the green photoelectric converter 40G, and the blue photoelectric converter 40B have light transmissivity, for example, the electrodes may be formed using any of the following materials. In a case where the electrode that may or may not have light transmissivity is an anode (for example, the lower electrode 15) having a function as an electrode extracting holes, the electrode is preferably formed using an electrically conductive material having a high work function (for example, ϕ=4.5 eV to 5.5 eV). Specific .examples of such a material include gold (Au), silver (Ag), chromium (Cr), nickel (Ni), palladium (Pd), platinum (Pt), iron (Fe), iridium (h), germanium (Ge), osmium (Os), rhenium (Re), tellurium (Te), and alloys thereof. In a case where the electrode that may or may not have light transmissivity is a cathode (for example, the upper electrode 17) having a function as an electrode extracting electrons, the electrode preferably includes an electrically conductive material having a low work function (for example, ϕ=3.5 eV to 4.5 eV). Specific examples of such a material include alkali metals (for example, Li, Na, K, and the like), fluorides thereof, and oxides thereof, alkali earth metals (for example, Mg, Ca, and the like), fluorides thereof, and oxides thereof, aluminum (Al), zinc (Zn), tin (Sn), thallium (Tl), an sodium-potassium alloy, an aluminum-lithium alloy, a magnesium-silver alloy, indium, and rare-earth metals such as ytterbium and alloys thereof.

In addition to the above-described materials, materials of the anode and the cathode include metals :such as platinum (Pt), gold (Au), palladium (Pd), chromium (Cr), nickel (Ni), aluminum (Al), silver (Ag), tantalum (Ta), tungsten (W), copper (Cu), titanium (Ti), indium (In), tin (Sn), iron (Fe), cobalt (Co), and molybdenum (Mo), alloys including these metal elements, and electrically conductive substances such as electrically conductive particles including these metals, :electrically conductive particles of alloys including these metals, polysilicon including an impurity, a carbon-based material, an oxide semiconductor, carbon nanotubes and graphene. The anode and the cathode may be configured as a single-layer film or a stacked film including the above-described elements. Further, as the materials included in the anode and the cathode, it is possible to use organic materials (electrically conductive polymers) such as poly(3,4-ethylenedioxythiophene)/polystyrene sulfonate [PEDOT/PSS]. In addition, these electrically conductive materials may be used for the electrodes by mixing the electrically conductive materials with a hinder (polymer) to form paste or an ink, and curing the paste or the ink.

In addition, in the above-described embodiment and the like, the configuration of the hack-side illumination type imaging apparatus has been exemplified; however, the contents of the present disclosure are applicable to a front-side illumination type imaging, apparatus. Further, the photoelectric conversion element of the present disclosure does not necessarily include all of the respective components described in the above-described embodiment, or, conversely, may include any other layer.

Furthermore, in the imaging element or the imaging apparatus, if necessary, a light-shielding layer may be provided, and a drive circuit or wiring for driving the imaging element may be provided. Furthermore, if necessary, a shutter for controlling entry of light to the imaging element may be provided, and an optical cut filter may be provided in accordance with the purpose of the imaging apparatus.

It is to be noted that the effects described herein are merely illustrative and non-limiting, and other effects may be included.

It is to be noted that the present disclosure may have the following configurations.
[1]

A photoelectric conversion element including:

a first electrode:
a second electrode opposed to the fist electrode; and
an organic layer provided between the first electrode and the second electrode, and including an organic photoelectric conversion layer,
at least one layer included in the organic layer being thrilled including at least one kind of organic semiconductor material represented by the following general expression (1).

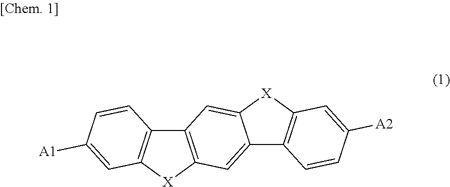

(X is one of an oxygen atom (O), a sulfur atom (S), and a selenium atom (Se), and A1 and A2 are each independently an aryl group, a heteroaryl group, an aryl amino group, a heteroaryl amino group, an aryl group having an aryl amino group as a substituent group, an aryl group having a heteroaryl amino group as a substituent group, a heteroaryl group having an aryl amino group as a substituent group a heteroaryl group having a heteroaryl amino group as a substituent group, or a derivative thereof).
[2]

The photoelectric conversion element according to [1], in which an aryl substituent group of the aryl group and the aryl amino group includes one of a phenyl group, a biphenyl group, a naphthyl group, a naphthyl phenyl group, a naphthyl biphenyl group, a phenyl naphthyl group, a tolyl group, a xylyl group, a terphenyl group, an anthracenyl group, a phenanthryl group, a pyrenyl group, a tetracenyl group, and a fluoranthenyl group.
[3]

The photoelectric conversion element according to [1], in which a heteroaryl substituent group of the heteroaryl group and the heteroaryl amino group includes one of a thienyl group, a thienyl phenyl group, a thienyl biphenyl group, a thiazoiyl group, a thiazolyl phenyl group, a thiazolyl biphenyl group an isothiazolyl group, an isothiazolyl phenyl group, an isothiazolyl biphenyl group, a furanyl group, a furanyl phenyl group, a furanyl biphenyl group, an oxazolyl group, an oxazolyl phenyl group, an oxazolyl biphenyl group, an oxadiazolyl group, an oxadiazolyl phenyl group, an oxadiazolyl biphenyl group, an isooxazolyl group, a benzothienyl group, a benzothienyl phenyl group, a benzothienyl biphenyl group, a benzofuranyl group, a pyrridinyl group, a pyridinyl phenyl group a biphenyl group, a quinolinyl group, a quinolyl phenyl group, a quinolyl biphenyl group, an isoquinolyl group, an isoquinolyl phenyl group, an isoquinolyl biphenyl group, an acridinyl group, an indole group, an indole phenyl group, an indole biphenyl group, an imidazole group, an imidazole phenyl group, an imidazole biphenyl group, a benzimidazole group, a benzimidazole phenyl group, a benzimidazole biphenyl group, and a carbazolyl group.

The photoelectric conversion element according to any one of [1] to [3], in which the organic photoelectric conversion layer is formed including the organic semiconductor material represented by the general expression (1).
[5]

The photoelectric conversion element according to any cine of [1] to [4], in which the organic semiconductor material represented by the general expression (1) includes a benzobisbenzothiophene derivative.

The photoelectric conversion element according to [5], in which the benzobisbenzothiophene derivative includes a compound represented by the following expression (1-1).

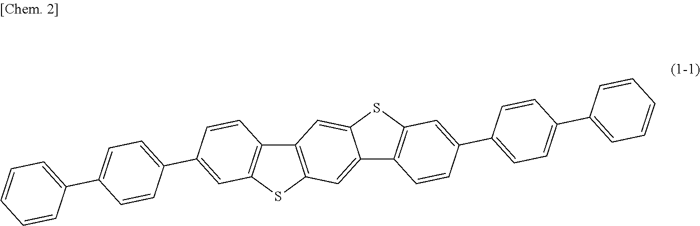 [SMILES: c1ccc(-c2ccc(-c3ccc4c(c3)sc3cc5c(cc34)sc3cc(-c4ccc(-c6ccccc6)cc4)ccc35)cc2)cc1]

The photoelectric conversion element according to [5], in which the benzobisbenzothiophene derivative includes a. compound represented by the following expression (1-2).

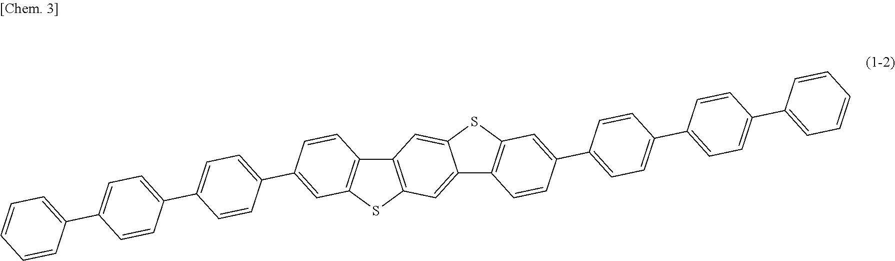 [SMILES: c1ccc(-c2ccc(-c3ccc(-c4ccc5c(c4)sc4cc6c(cc45)sc4cc(-c5ccc(-c7ccc(-c8ccccc8)cc7)cc5)ccc46)cc3)cc2)cc1]

[8]

The photoelectric conversion element according to any cine of [1] to [7], in Which the organic photoelectric conversion layer further includes at least one kind of fullerene C60 or a derivative thereof or fullerene C70 or a derivative thereof.
[9]

The photoelectric conversion element according to any one of [1] to [8], in which the organic photoelectric conversion layer further includes subphthalocyanine or a derivative thereof.
[10]

The photoelectric conversion element according to any one of [1] to [9], in Which the organic semiconductor material represented by the general expression (1) in a single-layer film having a film thickness of 5 nm to 100 nm both inclusive has a light absorptance of 0% to 3% both inclusive at a wavelength of 450 nm or greater, a light absorptance of 0% to 30% both inclusive at a wavelength of 425 nm, and a light absorptance of 0% to 80% both inclusive at a wavelength of 400 nm.
[1]

The photoelectric conversion element according, to any one of [4] to [10], in which an energy difference between an apparent HOMO level in the organic semiconductor material represented by the general expression (1) in the organic photoelectric conversion layer and a LUMO level of a material other than the organic semiconductor material represented by the general expression (1) in the organic photoelectric conversion layer is 1.1 eV or greater.
[12]

The photoelectric conversion element according to any one of [1] to [11], in which the first electrode and the second electrode each include a transparent electrically conductive material.
[13]

The photoelectric conversion element according to any one of [1] to [12], in which one of the first electrode and the second electrode includes a transparent electrically conductive material, and the other includes a metal material.
[14]

The photoelectric conversion element according to [13], in which the metal material includes one of aluminum (Al), an Al—Si—Cu alloy, and an Mg—Ag alloy.
[15]

The photoelectric conversion element according to any one of [1] to [14], in which

the organic layer includes any other layer in addition to the organic photoelectric conversion layer, and

the organic semiconductor material represented by the general expression (1) is included in the other layer.
[16]

An imaging apparatus provided with pixels each including one or a plurality of organic photoelectric converters, the organic photoelectric converters each including:

a first electrode;

a second electrode opposed to the first electrode; and

an organic layer provided between the first electrode and the second electrode, and including an organic photoelectric conversion layer,

at least one layer included in the organic layer being thrilled including at least one kind of organic semiconductor material represented by the following general expression (1).

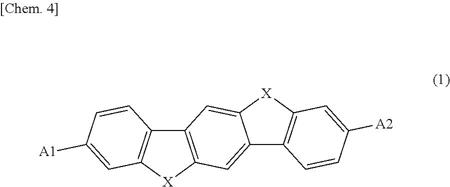

(X is one of an oxygen atom (O), a sulfur atom (S), and a selenium atom (Se), and A1 and A2 are each independently an aryl group, a heteroaryl group, an aryl amino group, a heteroaryl amino group, an aryl group having an aryl amino group as a substituent group, an aryl group having a heteroaryl amino group as a substituent group, a heteroaryl group having an aryl amino group as a substituent group a heteroaryl group having a heteroaryl amino group as a substituent group, or a derivative thereof.)
[17]

The imaging apparatus according to [16], in which one or a plurality of the organic photoelectric converters and one or a plurality of inorganic photoelectric converters that performs photoelectric conversion in a wavelength region different from the organic photoelectric converters are stacked in each of the pixels.
[18]

The imaging apparatus according to [16] or [17], in which a plurality of the organic photoelectric converters that performs photoelectric conversion in wavelength regions different from each other is stacked in each of the pixels.

This application claims the benefit of Japanese Priority Patent Application JP2017-215824 filed with the Japan Patent Office on Nov.8, 2017, the entire contents of which are incorporated herein by reference.

It should be understood by those skilled in the art that various modifications, combinations, sub-combinations, and alterations may occur depending on design requirements and other factors insofar as they are within the scope of the appended claims or the equivalents thereof.

What is claimed is:

1. A photoelectric conversion element comprising:
a first electrode;
a second electrode opposed to the first electrode; and
an organic layer provided between the first electrode and the second electrode, and including an organic photoelectric conversion layer,
at least one layer included in the organic layer being formed including at least one kind of organic semiconductor material represented by the following general expression (1). 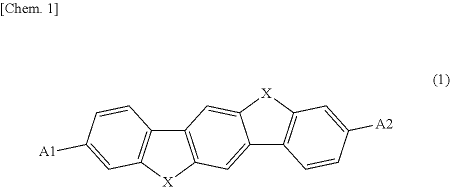
(X is one of an oxygen atom (O), a sulfur atom (S), and a selenium atom (Se), and A1 and A2 are each independently an aryl group, a heteroaryl group, an aryl amino group, a heteroaryl amino group, an aryl group having an aryl amino group as a substituent group, an aryl group having a heteroaryl amino group as a substituent group, a heteroaryl group having an aryl amino group as a substituent group, a heteroaryl group having a heteroaryl amino group as a substituent group, or a derivative thereof.)

2. The photoelectric conversion element according to claim 1, wherein an aryl substituent group of the aryl group and the aryl amino group includes one of a phenyl group, a biphenyl group, a naphthyl group, a naphthyl phenyl group, a naphthyl biphenyl group, a phenyl naphthyl group, a tolyl group, a xylyl group, a terphenyl group, an anthracenyl group, a phenanthryl group, a pyrenyl group, a tetracenyl group, and a fluoranthenyl group.

3. The photoelectric conversion element according to claim 1, wherein a heteroaryl substituent group of the heteroaryl group and the heteroaryl amino group includes one of a thienyl group, a thienyl phenyl group, a thienyl biphenyl group, a thiazolyl group, a thiazolyl phenyl group, a thiazolyl biphenyl group, an isothiazolyl group, an isothiazolyl phenyl group, an isothiazolyl biphenyl group, a furanyl group, a furanyl phenyl group, a furanyl biphenyl group, an oxazolyl group, an oxazolyl phenyl group, an oxazolyl biphenyl group, an oxadiazolyl group, an oxadiazolyl phenyl group, an oxadiazolyl biphenyl group, an isooxazolyl group, a benzothienyl group, a benzothienyl phenyl group, a benzothienyl biphenyl group, a benzofuranyl group, a pyridinyl group, a pyridinyl phenyl group, a pyridinyl biphenyl group, a quinolinyl group, a quinolyl phenyl group, a quinolyl biphenyl group, an isoquinolyl group, an isoquinolyl phenyl group, an isoquinolyl biphenyl group, an acridinyl group, an indole group, an indole phenyl group, an indole biphenyl group, an imidazole group, an imidazole phenyl group, an imidazole biphenyl group, a benzimidazole group, a benzimidazole phenyl group, a benzimidazole biphenyl group, and a carbazolyl group.

4. The photoelectric conversion element according to claim 1, wherein the organic photoelectric conversion layer is formed including the organic semiconductor material represented by the general expression (1).

5. The photoelectric conversion element according to claim 1, wherein the organic semiconductor material represented by the general expression (1) includes a benzobisbenzothiophene derivative.

6. The photoelectric conversion element according to claim 5, wherein the benzobisbenzothiophene derivative includes a compound represented by the following expression (1-1).

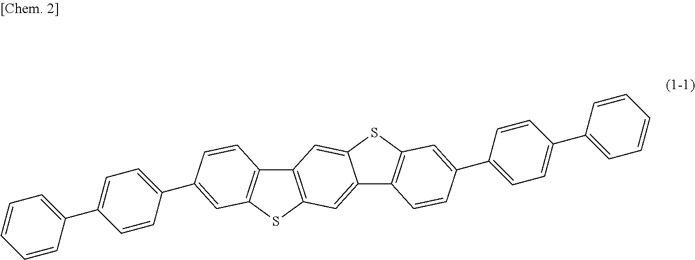 [SMILES: c1ccc(-c2ccc(-c3ccc4c(c3)sc3cc5c(cc34)sc3cc(-c4ccc(-c6ccccc6)cc4)ccc35)cc2)cc1]

7. The photoelectric conversion element according to claim 5, wherein the benzobisbenzothiophene derivative includes a compound represented by the following expression (1-2).

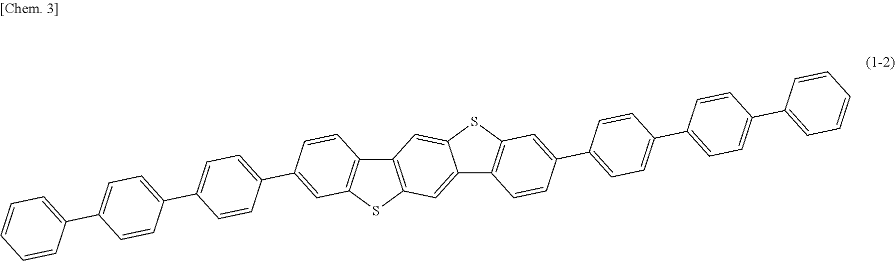 [SMILES: c1ccc(-c2ccc(-c3ccc(-c4ccc5c(c4)sc4cc6c(cc45)sc4cc(-c5ccc(-c7ccc(-c8ccccc8)cc7)cc5)ccc46)cc3)cc2)cc1]

8. The photoelectric conversion element according to claim 1, wherein the organic photoelectric conversion layer further includes at least one kind of fullerene C60 or a derivative thereof or fullerene C70 or a derivative thereof.

9. The photoelectric conversion element according to claim 1, wherein the organic photoelectric conversion layer further includes subphthalocyanine or a derivative thereof.

10. The photoelectric conversion element according to claim 1, wherein the organic semiconductor material represented by the general expression (1) in a single-layer film having a film thickness of 5 nm to 100 nm both inclusive has a light absorptance of 0% to 3% both inclusive at a wavelength of 450 nm or greater, a light absorptance of 0% to 30% both inclusive at a wavelength of 425 nm, and a light absorptance of 0% to 80% both inclusive at a wavelength of 400 nm.

11. The photoelectric conversion element according to claim 4, wherein an energy difference between an apparent HOMO level in the organic semiconductor material represented by the general expression (1) in the organic photoelectric conversion layer and a LUMO level of a material other than the organic semiconductor material represented by the general expression (1) in the organic photoelectric conversion layer is 1.1 eV or greater.

12. The photoelectric conversion element according to claim 1, wherein the first electrode and the second electrode each include a transparent electrically conductive material.

13. The photoelectric conversion element according to claim 1, wherein one of the first electrode and the second electrode includes a transparent electrically conductive material, and the other includes a metal material.

14. The photoelectric conversion element according to claim 13, wherein the metal material includes one of aluminum (Al), an Al—Si—Cu alloy, and an Mg—Ag alloy.

15. The photoelectric conversion element according to claim 1, wherein
the organic layer includes any other layer in addition to the organic photoelectric conversion layer, and
the organic semiconductor material represented by the general expression (1) is included in the other layer.

16. An imaging apparatus provided with pixels each including one or a plurality of organic photoelectric converters, the organic photoelectric converters each comprising:
a first electrode;
a second electrode opposed to the first electrode; and
an organic layer provided between the first electrode and the second electrode, and including an organic photoelectric conversion layer,
at least one layer included in the organic layer being formed including at least one kind of organic semiconductor material represented by the following general expression (1). 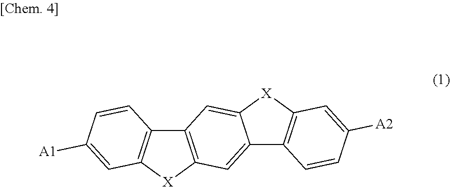
(X is one of an oxygen atom (O), a sulfur atom (S), and a selenium atom (Se), and A1 and A2 are each independently an aryl group, a heteroaryl group, an aryl amino group, a heteroaryl amino group, an aryl group having an aryl amino group as a substituent group, an aryl group having a heteroaryl amino group as a substituent group, a heteroaryl group having an aryl amino group as a substituent group, a heteroaryl group having a heteroaryl amino group as a substituent group, or a derivative thereof.)

17. The imaging apparatus according to claim 16, wherein one or a plurality of the organic photoelectric converters and one or a plurality of inorganic photoelectric converters that performs photoelectric conversion in a wavelength region different from the organic photoelectric converters are stacked in each of the pixels.

18. The imaging apparatus according to claim 16, wherein a plurality of the organic photoelectric converters that performs photoelectric conversion in wavelength regions different from each other is stacked in each of the pixels.
